(19) United States
(12) Patent Application Publication
(10) Pub. No.: US 2024/0131036 A1
(43) Pub. Date: Apr. 25, 2024

(54) COMPOSITIONS AND METHODS OF TREATING CONDITIONS

(71) Applicants: THE UAB RESEARCH FOUNDATION, Birmingham, AL (US); United States Government as Represented by the Department of Veterans Affairs, Washington, DC (US)

(72) Inventors: Andrzej T. SLOMINSKI, Birmingham, AL (US); Chander RAMAN, Birmingham, AL (US); Shariq QUAYYUM, Hoover, AL (US)

(21) Appl. No.: 18/004,069

(22) PCT Filed: Jul. 1, 2021
(86) PCT No.: PCT/US2021/040170

§ 371 Date: Jan. 2, 2023

Related U.S. Application Data
(60) Provisional application No. 63/047,546, filed on Jul. 2, 2020.
(60) Provisional application No. 63/180,715, filed on Apr. 28, 2021.

(51) Int. Cl. A61K 31/575; A61K 31/57; A61K 31/593; A61P 31/14

(52) U.S. Cl. CPC A61K 31/575; A61K 31/57; A61K 31/593; A61P 31/14

(57) ABSTRACT

The present disclosure provides for pharmaceutical composition and methods of treating a condition using the pharmaceutical composition. The condition to be treated in a subject in need of treatment can include an infection or non-viral hyper-inflammation/immune hyperactivation condition. The infection can be a coronavirus infection, HIV infection, influenza infection, or the like, where a direct or indirect result of the infection can be respiratory distress, oxidative stress, and the like that can lead to acute respiratory distress syndrome (ARDS) and/or organ dysfunction or failure.

### CROSS-REFERENCE TO RELATED APPLICATIONS

This application claims the benefit of and priority to U.S. Provisional Application Ser. No. 63/047,546, having the title “RESPIRATORY AND INTRANASAL DELIVERY OF ACTIVE FORMS OF VITAMIN D TO TREAT ACUTE RESPIRATORY DISTRESS SYNDROME”, filed on Jul. 2, 2020, and to U.S. Provisional Application Ser. No. 63/180,715, having the title “RESPIRATORY AND INTRANASAL DELIVERY OF ACTIVE FORMS OF VITAMIN D TO TREAT ACUTE RESPIRATORY DISTRESS SYNDROME” filed on Apr. 28, 2021, the disclosures of which are incorporated herein by reference in their respective entireties.

### FEDERAL FUNDING

This invention was made with government support under Grant Nos. RO1 AR073004-01A, 1 RO1 AR071189-01A1 and R21 A1149267-01, awarded by the National Institutes of Health and under Grant No. 1 I01 BX004293-01 awarded by the Veterans Administration. The government has certain rights in the invention.

### SEQUENCE LISTING

This application contains a sequence listing filed in electronic form as an ASCII.txt file entitled 222119_2960_ST25.bd, created on Jun. 30, 2021 and having a size of 2 KB. The content of the sequence listing is incorporated herein in its entirety.

### BACKGROUND

The Coronavirus disease 2019 (COVID-19) pandemic has brought tremendous socio-economic losses, causing great adversity with some intriguing and complex scientific questions to be answered. The variations in mortality and severity of severe acute respiratory syndrome coronavirus 2 (SARS-CoV-2) infected patients in different regions of the world is a challenging conundrum that still remains unanswered. The present disclosure addresses some of these challenges.

### SUMMARY

Embodiments of the present disclosure provide for pharmaceutical composition and methods of treating a condition using the pharmaceutical composition. In an aspect, the present disclosure provides for a pharmaceutical composition, comprising a therapeutically effective amount of an active agent or a pharmaceutically acceptable salt of the active agent, and a pharmaceutically acceptable carrier, to treat a condition, wherein the active agent is selected from the group consisting of: 9β,10α-cholesta-5,7-diene-3β,24-diol, 9β,10α-cholesta-5,7-diene-3β,25-diol, 9β,10α-cholesta-5,7-diene-3β,22-diol, 9β,10α-cholesta-5,7-diene-3β,20-diol, cholesta-5,7-dien-3β,20-diol, cholesta-5,7-dien-3β,22-diol, cholesta-5,7-dien-3β,25-diol, 3β-hydroxypregna-5,7-dien-20-one, (3S,5Z,7E)-9,10-secocholesta-5,7,10(19)-trien-3, 20-diol, (3S,5Z,7E)-9,10-secocholesta-5,7,10(19)-trien-3, 20, 23-triol, and optionally 9β,10α-cholesta-5,7-diene-3β,20,22-triol, 9β,10α-cholesta-5,7-diene-3β,27-diol, (3S,5Z,7E)-9,10-secocholesta-5,7,10(19)-trien-1,3,20-triol, and combinations thereof. The condition can be a COVID-19 condition or acute respiratory distress syndrome, for example. In an aspect, the pharmaceutical composition is in the form of an inhalant.

In an aspect, the present disclosure provides for a method of treating a condition comprising: administering to a subject in need thereof, a pharmaceutical composition, wherein the pharmaceutical composition includes a therapeutically effective amount of an active agent or a pharmaceutically acceptable salt of the active agent, and a pharmaceutically acceptable carrier, wherein the active agent is selected from the group consisting of: 9β,10α-cholesta-5,7-diene-3β,24-diol, 9β,10α-cholesta-5,7-diene-3β,25-diol, 9β,10α-cholesta-5,7-diene-3β,22-diol, 9β,10α-cholesta-5,7-diene-3β,20-diol, cholesta-5,7-dien-3β,20-diol, cholesta-5,7-dien-3β,22-diol, cholesta-5,7-dien-3β,25-diol, 3β-hydroxypregna-5,7-dien-20-one, (3S,5Z,7E)-9,10-secocholesta-5,7,10(19)-trien-3,20-diol, (3S,5Z,7E)-9,10-secocholesta-5,7,10(19)-trien-3,20, 23-triol, and optionally 9β,10α-cholesta-5,7-diene-3β,20,22-triol, 9β,10α-cholesta-5,7-diene-3β,27-diol, (3S,5Z,7E)-9,10-secocholesta-5,7,10(19)-trien-1, 3, 20-triol, and combinations thereof. In an aspect, administering can be via inhalation or parenteral.

In an aspect, the present disclosure provides for a pharmaceutical composition, comprising a therapeutically effective amount of an active agent or a pharmaceutically acceptable salt of the active agent, and a pharmaceutically acceptable carrier, to treat a COVID-19 condition, wherein the condition is a condition, wherein the COVID-19 agent is selected from the group consisting of: 9β,10α-cholesta-5,7-diene-3β,24-diol, 9β,10α-cholesta-5,7-diene-3β,25-diol, 9β,10α-cholesta-5,7-diene-3β,22-diol, 9β,10α-cholesta-5,7-diene-3β,20-diol, cholesta-5,7-dien-3β,20-diol, cholesta-5,7-dien-3β,22-diol, cholesta-5,7-dien-3β3,25-diol, 3β-hydroxypregna-5,7-dien-20-one, (3S,5Z,7E)-9,10-secocholesta-5,7,10(19)-trien-3,20-diol, (3S,5Z,7E)-9,10-secocholesta-5,7,10(19)-trien-3,20,23-triol, (3S,5Z,7E)-9,10-secocholesta-5,7,10(19)-trien-3,25 -diol, (3S,5Z,7E)-9,10-secocholesta-5,7,10(19)-trien-1,3,25-triol, (3S,5Z,7E)-9,10-secocholesta-5,7,10(19)-trien-1,3,20-triol, 9β,10α-cholesta-5,7-diene-3β,20,22-triol, 9β,10α-cholesta-5,7-diene-3β,27-diol and combinations thereof.

In an aspect, the present disclosure provides for a method of treating a COVID-19 condition comprising: administering to a subject in need thereof, a pharmaceutical composition, wherein the pharmaceutical composition includes a therapeutically effective amount of an active agent or a pharmaceutically acceptable salt of the active agent, and a pharmaceutically acceptable carrier, wherein the active agent is selected from the group consisting of: 9β,10α-cholesta-5,7-diene-3β3,24-diol, 9β,10α-cholesta-5,7-diene-3β,25-diol, 9β,10α-cholesta-5,7-diene-3β,22-diol, 9β,10α-cholesta-5,7-diene-3β3,20-diol, cholesta-5,7-dien-3β,20-diol, cholesta-5,7-dien-3β,22-diol, cholesta-5,7-dien-3β,25-diol, 3β-hydroxypregna-5,7-dien-20-one, (3S,5Z,7E)-9,10-secocholesta-5,7,10(19)-trien-3,20-diol, (3S,5Z,7E)-9,10-secocholesta-5,7,10(19)-trien-3,20,23-triol, (3S,5Z,7E)-9,10-secocholesta-5,7,10(19)-trien-3,25-diol, (3S,5Z,7E)-9,10-secocholesta-5,7,10(19)-trien-1, 3, 25-triol, (3S,5Z,7E)-9,10-secocholesta-5,7,10(19)-trien-1, 3,20-triol, 9β,10α-cholesta-5,7-diene-3β,20,22-triol, 9β,10α-cholesta-5,7-diene-3β,27-diol and combinations thereof.

Other compositions, methods, features, and advantages will be or become apparent to one with skill in the art upon examination of the following drawings and detailed description. It is intended that all such additional compositions, methods, features and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description, described below, when taken in conjunction with the accompanying drawings.

FIGS. 1.1A-1D illustrate the binding pattern of identified compounds with SARS-CoV-2 M^{pro}. FIG. 1.1A illustrates the structural representation of the protein in-complex with selected sterols and secosteroids. FIG. 1.1B illustrates the selected compounds blocking the binding pocket, and making significant interactions with the functionally important residues of SARS-CoV-2 M^{pro}. FIG. 1.1C illustrates the surface representation of conserved substrate-binding pocket of SARS-CoV-2 M^{pro} in complex with selected compounds. FIG. 1.1D illustrates a zoomed view of the substrate-binding pocket of SARS-CoV-2 M^{pro} in complexed with selected compounds.

FIGS. 1.2A-1.2C illustrate the binding pattern of identified sterols and secosteroids with SARS-CoV-2 RdRP. FIG. 1.2A illustrate the structural representation of the protein in complex with selected compounds. FIG. 1.2B illustrates the active site residues of the RdRP binding pocket making significant interactions with each of the identified compounds. FIG. 1.2C illustrates the surface view of the RdRP active site with the electrostatic potential from red (negative) to blue (positive) in complex with selected compounds.

FIG. 1.3A and 1.3B illustrate the enzyme inhibition by the selected sterols and secosteroids. FIG. 1.3A illustrates the M^{pro} enzyme inhibition by the selected metabolites at concentration of 2×10⁻⁷ M. The inhibition percentages were calculated using the formula: % inhibition=100×[1−(X−MINIMUM)/(MAXIMUM−MINIMUM)]. Min=negative control without any enzyme (0% enzyme activity), Max=positive control with enzyme and substrate (100% enzyme activity). The test sets included enzymes, substrates and the tests compound, excitation at a wavelength 360 nm and detection of emission at a wavelength 460 nm was observed for change in enzyme activity. The statistical significance of differences was evaluated by one-way ANOVA, *p<0.05, **p<0.01, ***p<0.001, for all conditions relative to the ethanol blank, n=3. FIG. 1.3B illustrates the RdRP enzyme activity inhibition by selected sterols and secosteroids. The inhibition percentages were calculated using the formula: % inhibition=100×[1−(X−MINIMUM)/(MAXIMUM−MINIMUM)]. Min=negative control without any enzyme (0% enzyme activity), Max=positive control with enzyme and substrate (100% enzyme activity). The statistical significance of differences was evaluated by one-way ANOVA, *p<0.05, **p<0.01, ***p<0.001, for all conditions relative to the ethanol blank, n=3

FIG. 1.4 illustrates 2D plots of the SARS-CoV-2 M^{pro} binding-pocket residues and their interactions with (A) 1,25(OH)₂D3, (B) 7DHP, (C) 20S(OH)7DHC, (D) 22R(OH)7DHC, (E) 25(OH)D3, (F) 25(OH)7DHC, (G) 20S(OH)D3, (H) A Michael acceptor inhibitor (N3).

FIG. 1.5 illustrates 2D plots of the SARS-CoV-2 RdRp binding-pocket residues and their interactions with (A) 1,25(OH)₂D3 (B) 20S(OH)L3, (C) 20S(OH)D3, (D) 22R(OH)7DHC, (E) 25(OH)D3 and (F) Remdesivir.

FIG. 2.1A-C illustrate that active forms of vitamin D and vitamin D itself are the solution to the COVID-19 illness. FIG. 2.1A illustrates novel and a classical hydroxy-derivatives of vitamin D₃, by inhibition of cytokine storm and oxidative stress, will attenuate ARDS (acute respiratory distress syndrome) and multiorgan failure induced by COVID-19. FIG. 2.1B illustrate the mechanism of action of canonical and non-canonical vitamin D-hydroxyderivatives. Vitamin D signaling in mononuclear cells downregulates inflammatory genes and suppresses oxidative stress. VDR—vitamin D receptor; RXR—retinoid X receptor; ROR—retinoic acid orphan receptor, RORE—retinoid orphan response element; ARE—antioxidant response element; VDRE—vitamin D response element; Nrf2—transcription factor NF-E2—related factor 2. FIG. 2.1C illustrates different routes of vitamin D delivery will impact vitamin D activation pattern.

FIG. 3.1 shows inhibition percentage observed in interaction of hACE2 and RBD in presence of the metabolites at concentration of 10-5 M.

FIG. 4.1 illustrates that lumisterol and vitamin D derivatives inhibit the expression of hACE2 in immortalized human HaCaT keratinocytes.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of” as well as other chemicals or minor impurities that are not active and do not impact treating the condition, in other words, the composition only includes the listed active agents but other non-active component can be present.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g. about 1%, about 2%, about 3%, and about 4%) and the sub-ranges (e.g. about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a pharmaceutical composition refers to an amount that is sufficient to achieve the desired improvement in the property modulated by the formulation component, e.g. achieving the desired level of treatment. The specific level in terms of wt % in a composition required as an effective amount will depend upon a variety of factors including the amount and type of pharmaceutical composition, the subject, severity of illness, and the like.

As used herein, the terms “optional” or “optionally” means that the subsequently described event or circumstance can or cannot occur, and that the description includes instances where said event or circumstance occurs and instances where it does not.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action, where the therapeutic agent can be present in a pharmaceutical composition. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used interchangeably herein, “subject,” “individual,” or “patient” can refer to a vertebrate organism, such as a bird, reptile, amphibian, mammal (e.g. human, canine, feline, equine, cattle, etc.). In an aspect, the subject is a human. In an aspect, the subject is a domesticated animal such as a dog or cat. “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as infections and consequences thereof. The effect can be therapeutic in terms of a partial or complete cure of a disease, condition, symptom or adverse effect attributed to the disease, disorder, or condition. The term “treatment” as used herein can include any treatment of infections such as COVID-19 infections (or other virus) and mutants and variants thereof in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration. In an aspect, the dosage for administering to a subject (e.g., a mammal, specifically a human) each active agent the present disclosure is about 2 to 60 micrograms/kilogram, where dosage can be adjusted as needed based on the active agent, the type of subject, the condition of the subject, the state of the disease or condition (e.g., COVID-19 condition or disease), and the like.

As used herein, “therapeutic” can refer to treating, healing, and/or ameliorating a disease, disorder, condition, or side effect, or to decreasing in the rate of advancement of a disease, disorder, condition, or side effect.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound (e.g., active agent) at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the disclosure (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound (e.g., active agent) and/or pharmaceutical composition (e.g., including the active agent), for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition (e.g., COVID-19 infection or other viral infection).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia.

In a particular aspect, administering refers administration non-orally. For example non-oral administration includes topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia.

The term “parenteral” can include subcutaneous, intravenous, intramuscular, intra-articular, intra-synovial, intrasternal, intrathecal, intralesional, and intracranial injections or infusion techniques.

In an aspect, administration is via inhalation. Administration can be continuous or intermittent. In various aspects, a composition or agent can be administered therapeutically; that is, administered to treat an existing disease or condition. In further various aspects, a composition or agent can be administered prophylactically; that is, administered for prevention of a disease or condition.

The disclosed composition (e.g., pharmaceutical composition including the active agent) or active agent can include those suitable for oral, rectal, topical, pulmonary, nasal, and parenteral administration (e.g., and in particular, rectal, topical, pulmonary, nasal, and parenteral administration), although the most suitable route in any given case will depend on the particular subject, and nature and severity of the conditions for which the active agent is being administered. In a further aspect, the disclosed composition (e.g., pharmaceutical composition) or agent can be formulated to allow administration orally, nasally, via inhalation, parenterally, transmucosally, transdermally, intramuscularly, intravenously, intradermally, subcutaneously, intraperitoneally, intraventricularly, intracranially and intratumorally (e.g., and in particular, nasally, via inhalation, parenterally, transmucosally, transdermally, intramuscularly, intravenously, intradermally, subcutaneously, intraperitoneally, intraventricularly, intracranially and intratumorally).

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1 -to-C 6 alkyl esters and C 5-to-C 7 cycloalkyl esters, although C 1 -to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1 -to-C 6 alkyl amines and secondary C 1 -to-C 6 dialkyl amines. In the case of secondary amines, the amine can also be in the form of a 5- or 6-membered heterocycle containing one nitrogen atom. Amides derived from ammonia, C 1 -to-C 3 alkyl primary amides and C 1 -to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, metabolites, esters, amides, salts of esters or amides, and N-oxides of a parent compound.

Unless otherwise specified, temperatures referred to herein are based on atmospheric pressure (i.e. one atmosphere).

### Discussion

Embodiments of the present disclosure provide for pharmaceutical composition and methods of treating a condition using the pharmaceutical composition. The pharmaceutical composition hydroxyforms of vitamin D and hydroxyderivatives of vitamin D and hydroxyklated forms thereof. The condition to be treated in a subject (e.g., mammal) in need of treatment can include an infection or non-viral hyper-inflammation/immune hyper-activation condition. The infection can be a coronavirus infection, HIV infection, influenza infection, hepatitis, or the like, where a direct or indirect result of the infection can be respiratory distress, oxidative stress, and the like that can lead to acute respiratory distress syndrome (ARDS) and/or organ dysfunction or failure. The active agent can act as an anti-inflammatory and/or anti-oxidative agent, which can disrupt, impair, terminate, downregulate, and the like the biological response and progress of the infection or non-viral hyper-inflammation/immune hyper-activation condition so as to treat (e.g., rescue) the subject. The method includes introducing the active agent an include inhalation and parenteral delivery to the subject. In addition, the active agent can be anti-hypertension, anti-atherogenic and inhibition atherosclerosis progression and anti-obesity properties, anti-allergic and anti-asthma activities. Additional details are provided in Examples 1 to 4.

Now having described aspect of the present disclosure briefly, additional details are now provided. The present disclosure provides for pharmaceutical compositions that can include a therapeutically effective amount of an active agent or a pharmaceutically acceptable salt of the active agent, and a pharmaceutically acceptable carrier, to treat a condition in a subject (e.g., human). In another aspect, the present disclosure provides for methods of treating a condition that includes administering to a subject in need thereof, a pharmaceutical composition, where the pharmaceutical composition includes a therapeutically effective amount of an active agent or a pharmaceutically acceptable salt of the active agent, and a pharmaceutically acceptable carrier. In an aspect, the therapeutically effective amount can be a dosage of 2 to 60 microgram/kilogram, where the amount can be adjusted with this range or outside of the range based on the type of subject, the active agent, the condition, age, health of the subject and the like.

In an aspect, the condition can be the direct or indirect result of a coronavirus infection (e.g., SARS-CoV-2 infection), other viruses infection, infectivity, or virally induced disease, e.g., HIV infection, influenza infection, hepatitis (e.g., A, B, C), virally induced diseases thereof and the like as well as non-viral hyper-inflammation/immune hyper-activation conditions, analgesic and antipyretic conditions. In addition, the condition can be type 1 diabetes, systemic lupus erythematosus, hepatocellular carcinoma, MHC class I complex, anti-metabolic syndrome. While not intending to be bound by theory, the active agent may be able to inhibit replication of the virus, inhibit the ability of the virus to infect cells, inhibit or terminate the cytokine storm, and the like. In a particular aspect, the condition can be a direct or indirect result of an infection caused by a coronavirus such as SARS-associated coronavirus (e.g., SARS-CoV or SARS-CoV-2). In one example, the condition can be acute respiratory distress syndrome (ARDS). In other examples, the condition can be organ damage due to oxidative stress and cytokine storm that may induced by infection or some other cause. In some instances the condition can be less severe but still require treatment where the condition is presented as a fever, cough, difficulty breathing, fatigue, muscle aches, headache, loss of taste or smell, congestion, nausea, diarrhea, and the like, where these conditions can worsen and/or lead to more serious conditions (e.g., pneumonia, dyspnea, hypoxia, respiratory failure, shock, organ(s) dysfunction or failure), where these can be the direct or indirect result of an infection and/or inflammation or immune condition. In an example, the condition is directly or indirectly caused by a COVID-19 infection. In another example, the condition is acute respiratory distress syndrome. In an aspect, the organ dysfunction or failure can impact the following organs: lungs, heart, brain, kidney, and the like.

In an aspect, the active agent is one or more of: 9β,10α-cholesta-5,7-diene-3β,24-diol, 9β,10α-cholesta-5,7-diene-3β,25-diol, 9β,10α-cholesta-5,7-diene-3β,22-diol, 9β,10α-cholesta-5,7-diene-3β,20-diol, cholesta-5,7-dien-3β,20-diol, cholesta-5,7-dien-3β,22-diol, cholesta-5,7-dien-3β,25-diol, 3β-hydroxypregna-5,7-dien-20-one, (3S,5Z,7E)-9,10-secocholesta-5,7,10(19)-trien-3,20-diol, and (3S,5Z,7E)-9,10-secocholesta-5,7,10(19)-trien-3,20,23-triol, and optionally (3S,5Z,7E)-9,10-secocholesta-5,7,10(19)-trien-1,3,20-triol, 9β,10α-cholesta-5,7-diene-3β,20,22-triol, and 9β,10α-cholesta-5,7-diene-3β,27-diol.

In another aspect, the active agent is one or more of: 9β,10α-cholesta-5,7-diene-3β,24-diol, 9β,10α-cholesta-5,7-diene-3β,25-diol, 9β,10α-cholesta-5,7-diene-3β,22-diol, 9β,10α-cholesta-5,7-diene-3β,20-diol, cholesta-5,7-dien-3β,20-diol, cholesta-5,7-dien-3β,22-diol, cholesta-5,7-dien-3β,25-diol, 3β-hydroxypregna-5,7-dien-20-one, (3S,5Z,7E)-9,10-secocholesta-5,7,10(19)-trien-3,20-diol, (3S,5Z,7E)-9,10-secocholesta-5,7,10(19)-trien-3,20,23-triol, (3S,5Z,7E)-9,10-secocholesta-5,7,10(19)-trien-3,25-diol, (3S,5Z,7E)-9,10-secocholesta-5,7,10(19)-trien-1,3,25-triol, (3S,5Z,7E)-9,10-secocholesta-5,7,10(19)-trien-1,3,20-triol, 9β,10α-cholesta-5,7-diene-3β,20,22-triol, and 9β,10α-cholesta-5,7-diene-3β,27-diol. Table 1 provides the chemical name and the associated chemical structure.

TABLE 1

| No. | Chemical name | abbreviation |
|---|---|---|
| 1. | 9β,10α-cholesta-5,7-diene- 3β,24-diol | 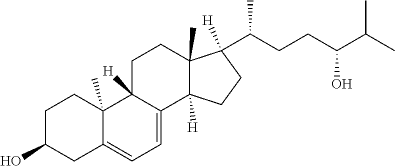 [SMILES: CC(C)[C@H](O)CC[C@@H](C)[C@H]1CC[C@H]2C3=CC=C4C[C@@H](O)CC[C@@]4(C)[C@@H]3CC[C@]12C] |
| 2. | 9β,10α-cholesta-5,7-diene- 3β,25-diol | 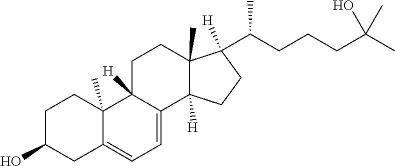 [SMILES: C[C@H](CCCC(C)(C)O)[C@H]1CC[C@H]2C3=CC=C4C[C@@H](O)CC[C@@]4(C)[C@@H]3CC[C@]12C] |
| 3. | 9β,10α-cholesta-5,7-diene- 3β,22-diol | 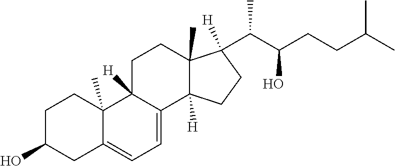 [SMILES: CC(C)CC[C@@H](O)[C@@H](C)[C@H]1CC[C@H]2C3=CC=C4C[C@@H](O)CC[C@@]4(C)[C@@H]3CC[C@]12C] |
| 4. | 9β,10α-cholesta-5,7-diene- 3β,20-diol | 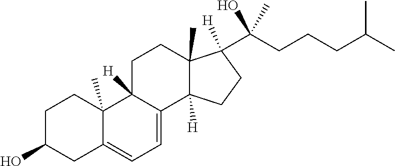 [SMILES: CC(C)CCC[C@](C)(O)[C@H]1CC[C@H]2C3=CC=C4C[C@@H](O)CC[C@@]4(C)[C@@H]3CC[C@@]21C] |
| 5. | Cholesta-5,7-dien-3β,20-diol | 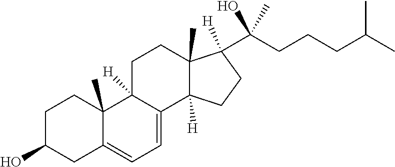 [SMILES: CC(C)CCC[C@](C)(O)[C@H]1CC[C@H]2C3=CC=C4C[C@@H](O)CC[C@]4(C)[C@H]3CC[C@@]21C] |
| 6. | Cholesta-5,7-dien-3β,22-diol | 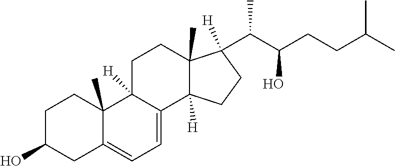 [SMILES: CC(C)CC[C@@H](O)[C@@H](C)[C@H]1CC[C@H]2C3=CC=C4C[C@@H](O)CC[C@]4(C)[C@H]3CC[C@]12C] |
| 7. | Cholesta-5,7-dien-3β,25-diol | 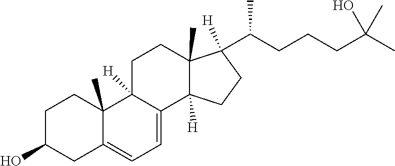 [SMILES: C[C@H](CCCC(C)(C)O)[C@H]1CC[C@H]2C3=CC=C4C[C@@H](O)CC[C@]4(C)[C@H]3CC[C@]12C] |
| 8. | 3β-hydroxypregna-5,7-dien- 20-one | 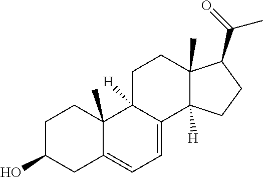 [SMILES: CC(=O)[C@H]1CC[C@H]2C3=CC=C4C[C@@H](O)CC[C@]4(C)[C@H]3CC[C@]12C] |
| 9. | (3S,5Z,7E)-9,10-secocholesta- 5,7,10(19)-trien-3, 20, 23-triol | 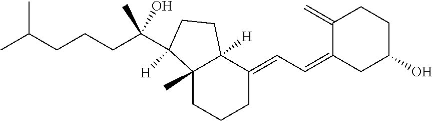 [SMILES: C=C1CC[C@H](O)C/C1=C/C=C1\CCC[C@@]2(C)[C@H]1CC[C@@H]2[C@@](C)(O)CCCC(C)C] |
| 10. | (3S,5Z,7E)-9,10-secocholesta- 5,7,10(19)-trien-3, 20, 23-triol | 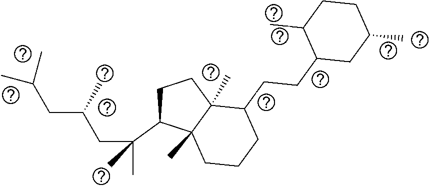 |
| 11. | (3S,5Z,7E)-9,10-secocholesta- 5,7,10(19)-trien-3, 25-diol | 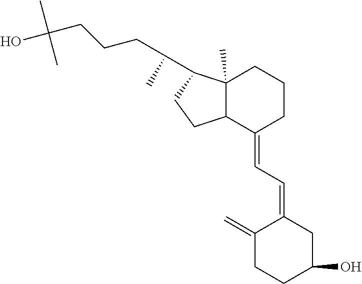 |
| 12. | (3S,5Z,7E)-9,10-secocholesta- 5,7,10(19)-trien-1, 3, 25-triol | 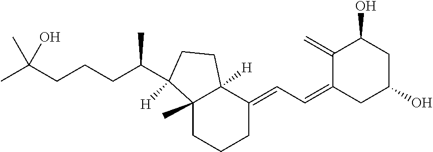 [SMILES: C=C1/C(=C\C=C2/CCC[C@]3(C)[C@@H]([C@H](C)CCCC(C)(C)O)CC[C@@H]23)C[C@@H](O)C[C@@H]1O] |
| 13. | (3S,5Z,7E)-9,10-secocholesta- 5,7,10(19)-trien-1, 3, 20-triol | 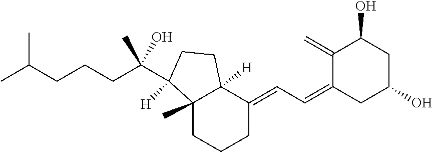 [SMILES: C=C1/C(=C\C=C2/CCC[C@@]3(C)[C@H]2CC[C@@H]3[C@@](C)(O)CCCC(C)C)C[C@@H](O)C[C@@H]1O] |
| 14. | 9β,10α-cholesta-5,7-diene- 3β,20,22-triol | 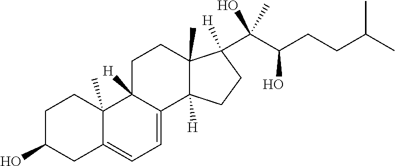 [SMILES: CC(C)CC[C@@H](O)[C@](C)(O)[C@H]1CC[C@H]2C3=CC=C4C[C@@H](O)CC[C@@]4(C)[C@@H]3CC[C@]12C] |
| 15. | 9β,10α-cholesta-5,7-diene- 3β,27-diol | 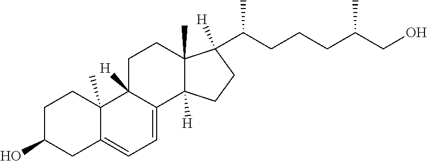 [SMILES: C[C@H](CO)CCC[C@@H](C)[C@H]1CC[C@H]2C3=CC=C4C[C@@H](O)CC[C@@]4(C)[C@@H]3CC[C@]12C] |

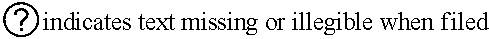

In regard to Table 1, compound no. 1 can also be referred to as 24-hydroxylumisterol, compound no. 2 can also be referred to as 25-hydroxylumisterol, compound no. 3 can also be referred to as 22-hydroxylumisterol, compound no. 4 can also be referred to as 20-hydroxylumisterol, compound no. 5 can also be referred to as 20-hydroxy-7-dehydrocholesterol, compound no. 6 can also be referred to as 22-hydroxy-7-dehydrocholesterol, compound no. 7 can also be referred to as 25-hydroxy-7-dehydrocholesterol, compound no. 8 can also be referred to as 7-dehydropregnenolone, compound no. 9 can also be referred to as 20-hydroxyvitamin D3, compound no. 10 can also be referred to as 20,23-dihydroxyvitamin D3, compound no. 11 can also be referred to as 25-hydroxyvitamin D3, compound no. 12 can also be referred to as 1,25-dihydroxyvitamin D3, compound no. 13 can also be referred to as 1,20-dihydroxyvitamin D3, compound no. 14 can also be referred to as 20, 22-dihydroxylumisterol, and compound no. 15 can also be referred to as 27-hydroxylumisterol (See Slominski A T, Janjetovic Z, Fuller B E, Zmijewski M A, Tuckey R C, et al. (2010) Products of vitamin D3 or 7-dehydrocholesterol metabolism by cytochrome P450scc show anti-leukemia effects, having low or absent calcemic activity. PLoS ONE 5(3): e990; Slominski A T, Kim T-K., Janjetovic Z, Tuckey R C, Bieniek, R, Yue Y, Li W, Chen J, Miller D, Chen T, Holick M (2011) 20-hydroxyvitamin D2 is a non-calcemic analog of vitamin D with potent antiproliferative and prodifferentiation activities in normal and malignant cells. Am J Physiol: Cell Physiol 300:C526-0541; Wang J, Slominski A T, Tuckey R C, Janjetovic Z, Kulkarni A, Chen J, Postlethwaite A, Miller D, Li W (2012) 20-Hydroxylvitamin D3 possesses high efficacy against proliferation of cancer cells while being non-toxic. Anticancer Res 32: 739-746, Slominski A, Janjetovic Z, Tuckey RC, Nguyen MN, Bhattacharya K G, Wang J, Li W, Jiao Y, Gu W, Brown M, Postlethwaite A E (2013) 20-hydroxyvitamin D3, noncalcemic product of CYP11A1 action on vitamin D3, exhibits potent antifibrogenic activity in vivo. J Clin Endocrinol Metab 98, E298-E30; Chen, J., J. Wang, T. Kim, E. Tieu, E. Tamg, Lin Z, D. Kovacic, D. Miller, A. Postlethwaite, R. Tuckey, A. Slominski and W. Li (2014). Novel Vitamin D Analogs as Potential Therapeutics: The Metabolism, Toxicity Profiling, and Antiproliferative Activity. Anticancer Res 34: 2153-2163.)

### Pharmaceutical Formulations and Routes of Administration

Embodiments of the present disclosure include the active agent as identified herein and can be formulated with one or more pharmaceutically acceptable excipients, diluents, carriers and/or adjuvants. In addition, embodiments of the present disclosure include the active agent formulated with one or more pharmaceutically acceptable auxiliary substances. In particular the active agent can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C . Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the active agent can be administered to the subject using any means capable of resulting in the desired effect. Thus, the active agent can be incorporated into a variety of formulations for therapeutic administration. For example, the active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the active agent may be administered in the form of its pharmaceutically acceptable salts, or a subject active composition may be used alone or in appropriate association, as well as in combination, with other pharmaceutically active compounds. The following methods and excipients are merely exemplary and are in no way limiting.

For oral preparations, the active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the active agent can be utilized in aerosol formulation to be administered via inhalation. Embodiments of the active agent can be formulated into pressurized acceptable propellants such as dichlorodifluoromethane, propane, nitrogen and the like.

Furthermore, embodiments of the active agent can be made into suppositories by mixing with a variety of bases such as emulsifying bases or water-soluble bases. Embodiments of the active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the active agent in a composition as a solution in sterile water, normal saline or another pharmaceutically acceptable carrier.

Embodiments of the active agent can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient (triamino-pyridine derivative and/or the labeled triamino-pyridine derivative) encapsulated in liposome vehicles in accordance with the present disclosure.

In an embodiment, the active agent can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the active agent can be accomplished using any of a variety of refillable, pump systems. Pumps provide consistent, controlled release over time. In some embodiments, the active agent can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, the active agent can be delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the active agent are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the active agent adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) can be delivered in a controlled release system. For example, the active agent may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) include those formed by impregnation of the active agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### Dosages

Embodiments of the active agent can be administered to a subject in one or more doses. Those of skill will readily appreciate that dose levels can vary as a function of the specific the active agent administered, the severity of the symptoms and the susceptibility of the subject to side effects. Preferred dosages for a given compound are readily determinable by those of skill in the art by a variety of means.

In an embodiment, multiple doses of the active agent are administered. The frequency of administration of the active agent can vary depending on any of a variety of factors, e.g., severity of the symptoms, and the like. For example, in an embodiment, the active agent can be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). As discussed above, in an embodiment, the active agent is administered continuously.

The duration of administration of the active agent, e.g., the period of time over the active agent is administered, can vary, depending on any of a variety of factors, e.g., patient response, etc. For example, the active agent in combination or separately, can be administered over a period of time of about one day to one week, about two weeks to four weeks, about one month to two months, about two months to four months, about four months to six months, about six months to eight months, about eight months to 1 year, about 1 year to 2 years, or about 2 years to 4 years, or more.

Dosage at concentrations as high as 60 micrograms/kilograms that are non-toxic. Also lower concentrations, such as 1-4 micrograms/kilogram, show biological activity in in vivo systems. The concentration in in vitro established at 10⁻⁹-10⁻⁶ M are active and this concentration is expected to be achieved in the cell environment. (See Slominski A T, Janjetovic Z, Fuller B E, Zmijewski M A, Tuckey R C, et al. (2010) Products of vitamin D3 or 7-dehydrocholesterol metabolism by cytochrome P450scc show anti-leukemia effects, having low or absent calcemic activity. PLoS ONE 5(3): e990; Slominski A T, Kim T-K., Janjetovic Z, Tuckey R C, Bieniek, R, Yue Y, Li W, Chen J, Miller D, Chen T, Holick M (2011) 20-hydroxyvitamin D2 is a non-calcemic analog of vitamin D with potent antiproliferative and prodifferentiation activities in normal and malignant cells. Am J Physiol: Cell Physiol 300:C526-0541; Wang J, Slominski AT, Tuckey RC, Janjetovic Z, Kulkarni A, Chen J, Postlethwaite A, Miller D, Li W (2012) 20-Hydroxylvitamin D₃ possesses high efficacy against proliferation of cancer cells while being non-toxic. Anticancer Res 32: 739-746; Slominski A, Janjetovic Z, Tuckey R C, Nguyen MN, Bhattacharya K G, Wang J, Li W, Jiao Y, Gu W, Brown M, Postlethwaite A E (2013) 20-hydroxyvitamin D3, noncalcemic product of CYP11A1 action on vitamin D3, exhibits potent antifibrogenic activity in vivo. J Clin Endocrinol Metab 98, E298-E30; Chen, J., J. Wang, T. Kim, E. Tieu, E. Tamg, Lin Z, D. Kovacic, D. Miller, A. Postlethwaite, R. Tuckey, A. Slominski and W. Li (2014). Novel Vitamin D Analogs as Potential Therapeutics: The Metabolism, Toxicity Profiling, and Antiproliferative Activity. Anticancer Res 34: 2153-2163.)

In an aspect, the dosage for administering to a subject (e.g., a mammal such as a human) having a condition (e.g., COVID-19) of any single active agent the present disclosure is about 2 to 60 micrograms/kilogram or a combination of active agents, each agent can be about 2 to 60 micrograms/kilogram.

### Routes of Administration

Embodiments of the present disclosure provide methods and compositions for the administration of the active agent to a subject (e.g., a human) using any available method and route suitable for drug delivery, including in vivo and ex vivo methods, as well as systemic and localized routes of administration.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An active agent can be administered in a single dose or in multiple doses.

Embodiments of the active agent can be administered to a subject using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the active agent. Where systemic delivery is desired, administration typically involves invasive or systemically absorbed topical or mucosal administration of pharmaceutical preparations.

In an embodiment, the active agent can also be delivered to the subject by enteral administration. Enteral routes of administration include, but are not limited to, oral and rectal (e.g., using a suppository) delivery.

Methods of administration of the active agent through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

The following examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

In brief, Example 1 describes molecular docking-based virtual screening studies predict that novel vitamin D and related lumisterol hydroxymetabolites are able to bind to the active sites of two SARS-CoV-2 transcription machinery enzymes with high affinity. These enzymes are the main protease (MP9 and RNA dependent RNA polymerase (RdRP) which play important roles in viral replication and establishing infection. Based on predicted binding affinities and specific interactions, we identified ten D3 and lumisterol analogs as likely binding partners of SARS-CoV-2 M^{pro} and RdRP and therefore tested their ability to inhibit these enzymes. Activity measurements demonstrated that 25(OH)L3, 24(OH)L3 and 20(OH)7DHC are the most effective of the hydroxymetabolites tested at inhibiting the activity of SARS-CoV-2 M^{pro}, causing 10-19% inhibition. These same derivatives as well as other hydroxylumisterols and hydroxyvitamin D3 metabolites inhibited RdRP by 50-60%. Thus, inhibition of these enzymes by vitamin D and lumisterol metabolites may provide a novel approach to hindering the SARS-CoV-2 infection.

In brief, Example 2 describes that active hydroxyl-forms of vitamin D are anti-inflammatory, induce anti-oxidative responses, and stimulate innate immunity against infectious agents. These properties are shared by calcitriol and the CYP11A1-generated non-calcemic hydroxyderivatives. They inhibit the production of pro-inflammatory cytokines, downregulate NF-κB, show inverse agonism on RORγ and counteract oxidative stress through the activation of NRF-2. Therefore, a direct delivery of hydroxyderivatives of vitamin D deserves consideration in the treatment of COVID-19 or ARDS of different etiology. We also recommend treatment of COVID-19 patients with high dose vitamin D since populations most vulnerable to this disease are likely vitamin D deficient and patients are already under supervision in the clinics. We hypothesize that different routes of delivery (oral and parenteral) will have different impact on the final outcome

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g. amounts, temperature, etc.), but some errors and deviations should be accounted for. Unless indicated otherwise, parts are parts by weight, temperature is in ° C. or is at ambient temperature, and pressure is at or near atmospheric.

### Example 1

The Coronavirus disease 2019 (COVID-19) pandemic has brought tremendous socio-economic losses, causing great adversity with some intriguing and complex scientific questions to be answered. The variations in mortality and severity of severe acute respiratory syndrome coronavirus 2 (SARS-CoV-2) infected patients in different regions of the world is a challenging conundrum that still remains unanswered. A plethora of reports has made a striking and intriguing co-relation that vitamin D deficiency influences the risk of mortality in SARS-CoV-2 infected patients [1]. Vitamin D is a pleotropic hormone, which exerts genomic and non-genomic effects on expression of a large number of genes that modify many important biological functions [2]. The genomic effects initially involve the binding of 1,25(OH) 2 D3 (1,25-dihydroxyvitamin D3) to the VDR (vitamin D receptor). The ligand-based activation of this nuclear transcription factor can modulate the expression of genes positively or negatively. However, a variety of signaling pathways can be activated in a non-genomic manner by 1,25(OH)₂D3 [2]. Multiple mechanisms have been proposed by which vitamin D protects against infection and reduces mortality when present in sufficient physiological levels [3-5]. Vitamin D modulates the innate and adaptive immune response [6] and hence vitamin D deficiency is a recognized risk factor for the cytokine storm and ARDS (Acute respiratory distress syndrome) (reviewed in [7, 8]). Vitamin D also has been reported to affect the expression of ACE2 and TMPRSS2, membrane receptors responsible for the cellular entry of the virus [9]. In addition, active forms of vitamin D can regulate the expression of the antiviral pathways and also viral load in the system, when used at high concentrations [5, 10]. However, as of 2021, there has been only one report confirming a direct relationship between vitamin D and inhibition of the SARS-CoV-2 viral replication where it was shown that calcitriol (1,25(OH)₂D3) reduces the viral

load in the CPE (Cytopathogenic effect) assay [11]. Although the authors did not provide the mechanism behind this action, their study indicates that active forms of vitamin D3 (D3) may have the potential to inhibit or neutralize SARS-CoV-2 growth and reduce the severity of the infection.

The exposure of skin to ultraviolet radiation (UVB) leads to conversion of 7DHC to previtamin D3 (pre-D3) which then undergoes thermal isomerization to form D3 [6, 12]. With a high UVB dose, pre-D3 undergoes photoisomerization to lumisterol (L3) and tachysterol (T3). T3, being the most photoreactive product, undergoes UVB-driven conversion to L3 via pre-D3. The resulting product, L3, is the major photoisomer observed in skin after prolonged UVB

exposure [13]. Traditionally, only D3 was regarded as the important biological regulator from these photochemical reactions, while L3 was considered to not affect calcium metabolism nor

display any other significant biological activity [14]. It was postulated that transformation of pre-D3 into metabolically inactive L3 explained why UVB-induced cutaneous production of pre-D3

does not lead to systemic D3 intoxication [12]. It is now apparent that this assumption is erroneous since new CYP11A1- and CYP27A1-dependant pathways of hydroxylation of L3 have been discovered with the products such as 20(OH)L3, 22(OH)L3, 24(OH)L3 and 25(OH)L3 displaying biological activity [14-17]. CYP11A1-mediated pathways of of both L3 and vitamin D metabolism have been shown to occur in vivo with their products having phenotypic/biological activities determined by their structures and cellular targets [14].

To evaluate whether these novel L3 and D3 hydroxyderivatives possess anti-viral potential, we screened them for effects on viral replication and transcription machinery proteins that are the most promising drug targets against SARS-CoV-2. The proteins tested in the study were RNA-dependent RNA polymerase (RdRP or nsp12) and 3C-like protease (3CLP r° or M^{pro}). SARS-CoV-2 contains a —30-kb RNA genome encoding two large overlapping polyprotein precursors (pp1a and pp1ab), four structural proteins (spike, envelope, membrane, and nucleocapsid), and several accessory proteins [18, 19]. It is essential for SARS-CoV-2 replication that the two polyproteins (pp1a/pp1ab) be cleaved into individual nonstructural proteins. M^{pro} exclusively cleaves polypeptides after a glutamine (Gin) residue and no other human protease have the same cleavage specificity as M^{pro} [19, 20]. This critical function of SARS-CoV-2 M^{pro} in replication and transcriptional regulation has made it a prime target for drug discovery purposes [18, 19]. It shows a 96% similarity at the amino acid sequence level with SARS-CoV M^{pro} [20, 21]. RdRP is another conserved protein of retroviruses and is also a

proven target for the development of antiviral drugs [22]. RdRP has two binding sites for ligands, one is the active site which binds nucleoside inhibitors (NIs) and the other is an alosteric site that binds non-nucleoside inhibitors (NNIs). Remdesivir mimics a nucleoside triphosphate substrate for the polymerase becoming covalently linked to the replicating RNA which interferes with further synthesis of the RNA [23].

In this example, we used a systematic approach for structure-based drug discovery in the search for potential therapeutic candidates from a pool of vitamin D and lumiterol metabolites, targeting the SARS-CoV-2 replication machinery enzymes M^{pro} and RdRP. Structure-based virtual screening was used to predict analogs that could bind to these two proteins followed by enzyme inhibition studies to confirm these interactions.

### Materials and Methods

### Materials

Methods of production of the compounds tested in this study and their structures are described in Table S1.

### Structure-Based Virtual Screening

In this study, a systematic in silico approach was utilized using a number of bioinformatics software, such as MGL AutoDock Tools [22], AutoDock Vina and Discovery Studio Visualizer [24]. The three-dimensional coordinates of SARS-CoV-2 M^{pro} and RdRP were retrieved from the RCSB Protein Data Bank (PDB). The structure of SARS-CoV-2 M^{pro} in complex with a Michael acceptor inhibitor known as N3 provides a basis to design high-affinity

inhibitors with improved pharmacological properties [17]. The atomic coordinates of SARS-CoV-2 M^{pro} were taken from the PDB (ID: 6LU7) for molecular docking-based virtual screening. The structural coordinates of SARS-CoV-2 RdRP in apo form and in complex with Remdesivir were taken from the PDB (ID: 7BV1 and 7BV2, respectively). The structure was prepared by removing co-crystallized inhibitors and adding hydrogens, and assigning appropriate atom types. AutoDock Vina was used to carry out molecular docking, where the search space was structurally blind for all the compounds with default docking parameters. High-affinity compounds were selected, and their docked conformations were generated for analyzing their possible interaction towards SARS-CoV-2 M^{pro} and RdRP. Only those compounds which specifically interact with the critical residues of the binding pockets of SARS-CoV-2 M^{pro} and RdRP were selected from the interaction analysis.

### Enzymatic Assay for M^{pro}

The SARS-CoV-2 3CL^{Pro} (M^{pro}), MBP-tagged Assay Kit (BPS Biosciences, San Diego, CA, USA) was used to measure 3CL Protease (M^{pro}) activity for screening and profiling applications. The 3CL inhibitor GC376 was included as a positive control and also used as a standard. The assay was performed according to the protocol provided by the manufacturer with the concentration of compounds tested being 10⁻⁷ M.

### Enzymatic Assay for RdRP

The assay for RdRP activity was outsourced to BPS Biosciences (San Jose, California, USA) using the following protocol. Dilutions of the compounds tested were prepared in assay buffer containing 5% ethanol, then 2 μl of this was added to the reaction mix to give a final volume of 10 μl, thus making the final ethanol concentration 1% in all reactions (controls without compounds similarly contained 1% ethanol). The RdRP reactions were performed in triplicate at 37° C. for 60 min in a 10 μl mixture containing assay buffer, RNA duplex, ATP substrate and enzyme, and the test compound. The reactions were carried out in wells of 384-well Optiplate (PerkinElmer). After enzymatic reactions, 10 μl of anti-Dig Acceptor beads (PerkinElmer, diluted 1:500 with lx detection buffer) were added to the reaction mix. After brief shaking, the plate was incubated for 30 min. Finally, 10 μl of AlphaScreen Streptavidin-conjugated donor beads (Perkin, diluted 1:125 with lx detection buffer) were added. After 30 min, the samples were measured using an AlphaScreen microplate reader (EnSpire Alpha 2390 Multilabel Reader, Perkin Elmer). The values of % activity versus the concentrations of the compound tested were then plotted using non-linear regression analysis of a Sigmoidal dose-response curve generated with the equation Y=B+(T−B)/1+10^{((LogEC50-X)×Hill Slope)}, where Y=percent activity, B=minimum percent activity, T=maximum percent activity, X=logarithm of compound and Hill Slope=slope factor or Hill coefficient. The IC₅₀ value was determined as the concentration causing half-maximal activity.

### Results and Discussion

Structure-based virtual screening of an in-house library of D3, L3 and 7DHC analogs was performed to search for high-affinity binding partners of SARS-CoV-2 M^{pro} and RdRP that

could be used in the design of potential therapeutics against COVID-19 [24]. We first calculated the binding affinities of the available compounds in the library when bound to SARS-CoV-2 ^{pro} and RdRP, and then performed interaction analysis to identify better hits. Based on the specific interaction, we identified a set of ten D3, L3 and 7DHC derivatives out of 35 compounds tested that had substantial affinity and specific interactions with the active site pocket of SARS-CoV-2 M^{pro} and RdRP (Table 1a & 1b). The predicted binding affinities of the 10 compounds compared well with those of recognized anti-virals, Danoprevir, Lopinavir and Ritonavir (Table 1a & 1b).

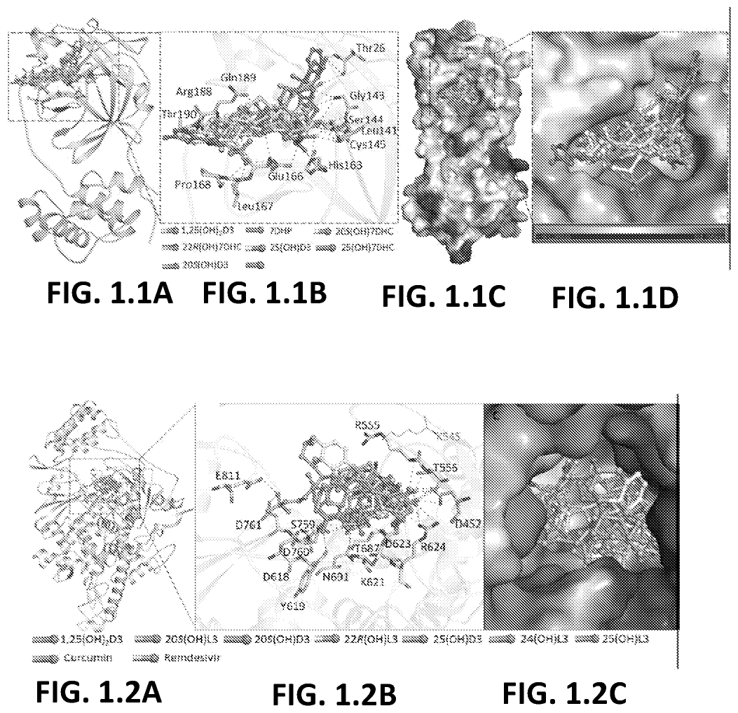

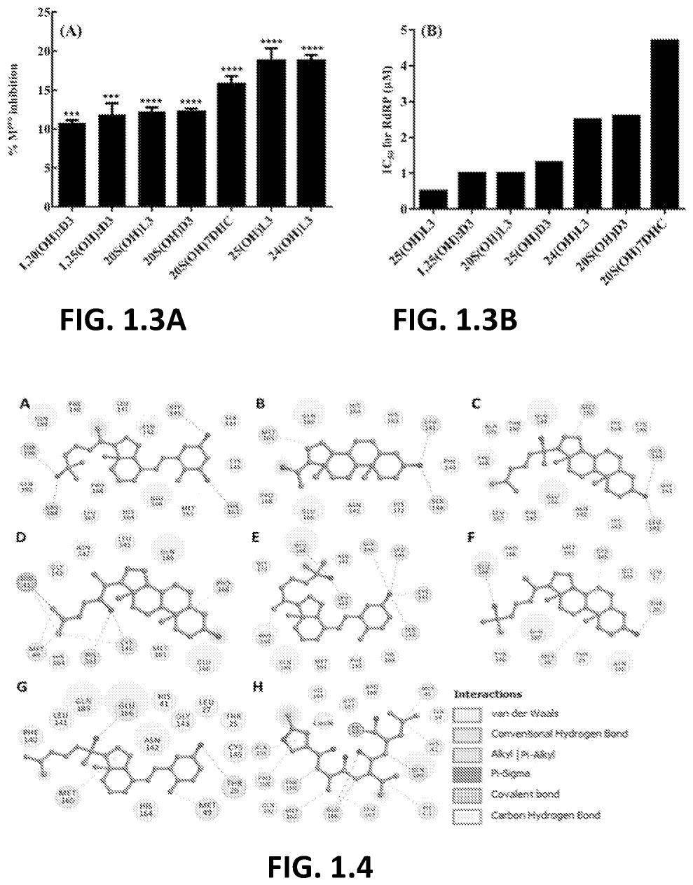

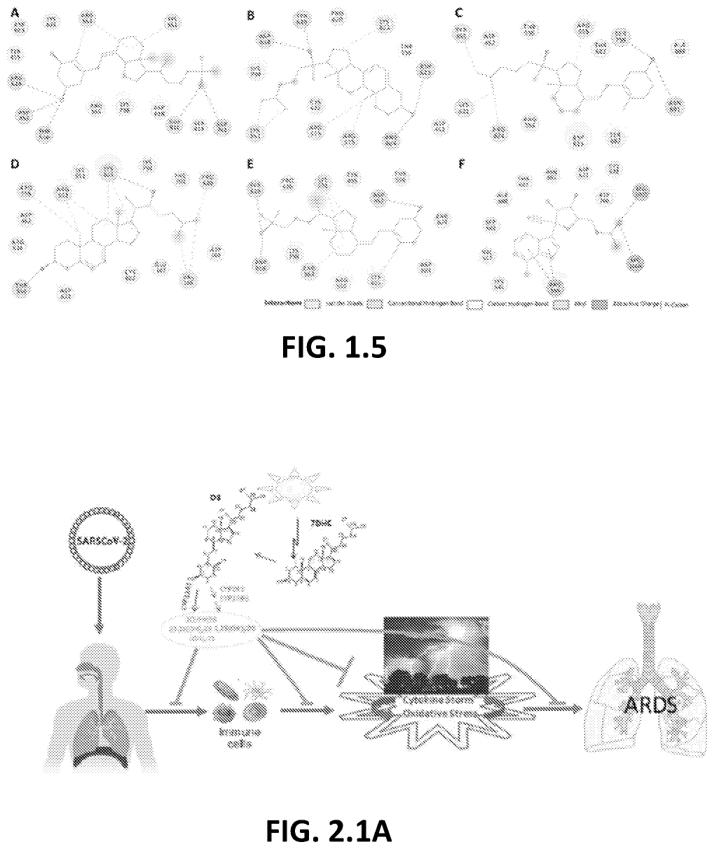

A detailed analysis of all the docked conformers of the top ten hits was performed to find compounds binding specifically to the SARS-CoV-2 M^{pro} and RdRP substrate-binding pockets (FIGS. 1.4 and 1.5). We selected seven compounds that form interactions to a set of critical residues of SARS-CoV-2 M^{pro} to study in detail. The critical active site residue Cys145 of SARS-CoV-2 M^{pro} along with the bonding pocket residues Thr26, His41, Leu141, Asn142, Gly143, Ser144, Cys145, His163, Glu166, Leu167, Pro168, Arg188, GIn189 and Thr190, provide a significant number of interactions with each of the seven compounds (FIGS. 1.1 and 1.4). Importantly, all of these sterols and secosteroids mimic the same binding pose and show a similar pattern as N3, a co-crystallized inhibitor of SARS-CoV-2 M^{pro}. The binding pattern of the selected compounds indicates a virtuous complementarity with the M^{pro} binding pocket which may hinder the substrate accessibility, thus inhibiting the enzymatic activity. Each of the seven compounds make significant interactions with critically important residues of the M^{pro} substrate-binding pocket (FIG. 1.1B and 1.4) and blocks the substrate-binding pocket of COVID-19 M^{pro} (FIG. 1.1C and 1.1D). The compounds docked to the M^{pro} binding pocket were analyzed further for their detailed interactions with the critical residues, including Cys145 (FIG. 1.4) [18].

Similar to M^{pro} molecular docking, we selected top 10 hits based on interaction with the SARS-CoV-2 RdRP active site pocket, from which we identified seven compounds that form interactions with a set of critically essential residues of SARS-CoV-2 RdRP. The residues Lys545, Arg555, Asp623, Ser682, Thr687, Asn691, Ser759, Asp760, and Asp761 of SARS-CoV-2 in RdRP substrate binding pocket that interact with Remdesivir, also offered a significant number of interactions with each of the seven compounds (FIGS. 1.2 and 1.5). Notably, these sterols and secosteroids have the same binding pattern as reported for the inhibitor Remdesivir with SARS-CoV-2 RdRP [23]. The binding prototype of the compounds indicates a virtuous complementarity to the SARS-CoV-2 RdRP binding pocket indicating that they have the capability to inhibit its enzymatic activity (FIG. 1.2). The compounds, including curcumin, interact with residues within RdRP active site pocket (FIG. 1.2A-B and 1.5) that are critical for substrate-binding (FIG. 1.2C).

The binding data predict that the selected sterols and secosteroids are likely to function as inhibitors of M^{pro} and RdRP enzyme activity and potentially serve as therapeutics against

COVID-19. Consistent with this prediction, we found that the compounds inhibited M^{pro} enzyme activity using the 3CL Protease, MBP-tagged (SARS-CoV-2) Assay kit (FIG. 1.3A). The results show that all 7 sterols and secosteroids tested inhibited M^{pro} activity with 25(OH)L3, 24(OH)L3 and 20S(OH)7DHC being most effective, inhibiting it by 10-19% at a concentration of 2×10⁻⁷ M (FIG. 1.3A).

RdRP catalyzes the replication of RNA from an RNA template and it is known that SARS-CoV-2 RdRP only functions when all three subunits are present (nsp12, nsp7 & nsp8). Hence, establishing the assay to measure its activity was difficult so this was outsourced to BPS biosciences who had already developed an in-house assay to measure enzyme activity. Although, the inhibition of RdRP activity did not precisely reflect the pattern of docking energies, all of the compounds tested exhibited inhibitory activity ranging from 40-60% at a concentration of 2×10⁻⁷ M (FIG. 1.3B). The IC₅₀ was also calculated for each compound which revealed that the most potent was 25(OH)L3 with an IC₅₀ of 0.5 μM followed by 1,25(OH)₂D3 and 20S(OH)L3 which had an IC₅₀ of 1 μM (Table S2).

Overall, these results provide strong support for the ability of D3, L3 and 7DHC hydroxy-metabolites to reduce the viral load in infected cells or the blood stream. A deficiency of these hydroxyometabolites may play a vital role in enabling the transition of SARS-CoV-2 patients from becoming asymptomatic to symptomatic. For M^{pro}, 25(OH)D3, the major form of vitamin D3 present in blood, significant inhibition was observed at a concentration of 100 nM which compares to a plasma concentration in non-deficient people typically between 50 to 100 nM [15]. For RdRP, the IC₅₀ for 25(OH)D3 was 1.3 μM, approximately one order of magnitude above its plasma concentration. For the hydroxylumisterols tested, plasma concentrtions are

unknown except for 20(OH)L3 where a value of 25 nM has been reported [17], but based on the enzymology, 25(OH)L3 which had the lowest IC₅₀ (0.5 μM) for inhibition of RdRP, is likely to be substantially higher [15].

A plethora of reports strongly suggests that vitamin D plays a vital role in protection against SARS-COV-2 which includes preventing infected patients from developing severe disease. Here, we report for the first time that a range of vitamin D3 related compounds, including 7DHC and L3 hydroxyderivaties, display anti-SARS-CoV-2 activities of and we provide a possible target on which they may act directly. Vaccines against SARS-CoV-2 are clearly a major advance in controlling COVID-19, however, new viral variants emphasize the need for alternative therapeutic approaches. This study presents novel vitamin D and L3 metabolites as candidates for anti-viral drugs.

TABLE 1
Binding affinities of vitamin D and lumisterol
derivatives to the SARS-COV-2 enzymes.

| | | |
|---|---|---|
| | | Binding Free Energy |
| No. | Name of the ligand | (kcal/mol) |

a: Binding affinities to the SARS-COV-2 M^{pro} in comparison to danoprevir,
lopinavir and ritonavir.

| | | |
|---|---|---|
| 16. | 24(OH)L3 | −8.3 |
| 17. | 25(OH)L3 | −8.0 |
| 18. | 20S(OH)7DHC | −8.0 |
| 19. | 22R(OH)7DHC | −7.8 |
| 20. | 20S(OH)L3 | −7.8 |
| 21. | 25(OH)7DHC | −7.7 |
| 22 | 1,25(OH)2D3 | −7.6 |
| 23. | 20S(OH)D3 | −7.6 |
| 24. | 7DHP | −7.5 |
| 25 | 25(OH)D3 | −7.3 |
| 26 | Danoprevir | −8.5 |
| 27. | Lopinavir | −8.3 |
| 28. | Ritonavir | −7.2 |

b: Binding affinities of to the SARS-COV-2 RdRP in comparison to ritonavir, danoprevir,
lopinavir and remdesivir.

| | | |
|---|---|---|
| 1. | 25(OH)D3 | −8.5 |
| 2. | 20S(OH)D3 | −8 |
| 3. | Ritonavir | −8 |
| 4. | Danoprevir | −7.9 |
| 5. | 20S(OH)L3 | −7.8 |
| 6. | 25(OH)L3 | −7.7 |
| 7. | 1,25(OH)2D3 | −7.7 |
| 8. | 20S(OH)7DHC | −7.6 |
| 9. | 22R(OH)L3 | −7.5 |
| 10. | 24(OH)L3 | −7.5 |
| 11. | 25(OH)7DHC | −7.5 |
| 12. | Lopinavir | −7.3 |
| 13. | Remdesivir | −7.3 |

### References for Example 1

1. Maghbooli, Z., Sahraian, M. A., Ebrahimi, M., Pazoki, M., Kafan, S., Tabriz, H. M., Hadadi, A., Montazeri, M., Nasiri, M. & Shirvani, A. (2020) Vitamin D sufficiency, a serum 25-hydroxyvitamin D at least 30 ng/mL reduced risk for adverse clinical outcomes in patients with COVID-19 infection, PloS one. 15, e0239799.
2. Haussler, M. R., Jurutka, P. W., Mizwicki, M. & Norman, A. W. (2011) Vitamin D receptor (VDR)-mediated actions of 1a, 25 (OH) 2vitamin D3: genomic and non-genomic mechanisms, Best practice & research Clinical endocrinology & metabolism. 25, 543-559.
3. Grant, W. B., Al Anouti, F. & Moukayed, M. (2020) Targeted 25-hydroxyvitamin D concentration measurements and vitamin D3 supplementation can have important patient and public health benefits, Eur J Clin Nutr. 74, 366-376.
4. Shirvani, A., Kalajian, T. A., Song, A. & Holick, M. F. (2019) Disassociation of Vitamin D′s Calcemic Activity and Non-calcemic Genomic Activity and Individual Responsiveness: A Randomized Controlled Double-Blind Clinical Trial, Sci Rep. 9, 17685.
5. Teymoori-Rad, M., Shokri, F., Salimi, V. & Marashi, S. M. (2019) The interplay between vitamin D and viral infections, Reviews in Medical Virology. 29, e2032.
6. Bikle, D. D. (2020) Vitamin D: Newer Concepts of Its Metabolism and Function at the Basic and Clinical Level, J Endocr Soc. 4, bvz038.
7. Quesada-Gomez, J. M., Entrenas-Castillo, M. & Bouillon, R. (2020) Vitamin D receptor stimulation to reduce acute respiratory distress syndrome (ARDS) in patients with coronavirus SARS-CoV-2 infections: Revised Ms SBMB 2020_166, The Journal of steroid biochemistry and molecular biology. 202, 105719.
8. Slominski, R. M., Stefan, J., Athar, M., Holick, M. F., Jetten, A. M., Raman, C. & Slominski, A. T. (2020) COVID-19 and Vitamin D: A lesson from the skin, Exp Dermatol. 29, 885-890.
9. Rhodes, J. M., Subramanian, S., Laird, E., Griffin, G. & Kenny, R. A. (2021) Perspective: Vitamin D deficiency and COVID-19 severity-plausibly linked by latitude, ethnicity, impacts on cytokines, ACE2 and thrombosis, Journal of internal medicine. 289, 97-115.
10. Coussens, A. K., Naude, C. E., Goliath, R., Chaplin, G., Wilkinson, R. J., & Jablonski, N. G. (2015) High-dose vitamin D3 reduces deficiency caused by low UVB exposure and limits HIV-1 replication in urban Southern Africans. Proceedings of the National Academy of Sciences, 112, 8052-8057.
11. Mok, C. K., Ng, Y. L., Ahidjo, B. A., Lee, R. C. H., Loe, M. W. C., Liu, J., Tan, K. S., Kaur, P., Chng, W. J. & Wong, J. E. L. (2020) Calcitriol, the active form of vitamin D, is a promising candidate for COVID-19 prophylaxis, bioRxiv.
12. Holick, M. F. (2003) Vitamin D: A millenium perspective, J Cell Biochem. 88, 296-307.
13. Zu, S., Deng, Y.-Q., Zhou, C., Li, J., Li, L., Chen, Q., Li, X.-F., Zhao, H., Gold, S. & He, J. (2020) 25-Hydroxycholesterol is a potent SARS-CoV-2 inhibitor, Cell research. 30, 1043-1045.
14. Slominski, A. T., Chaiprasongsuk, A., Janjetovic, Z., Kim, T. K., Stefan, J., Slominski, R. M., Hanumanthu, V. S., Raman, C., Qayyum, S., Song, Y., Song, Y., Panich, U., Crossman, D. K., Athar, M., Holick, M. F., Jetten, A. M., Zmijewski, M. A., Zmijewski, J. & Tuckey, R. C. (2020) Photoprotective Properties of Vitamin D and Lumisterol Hydroxyderivatives, Cell Biochem Biophys. 78, 165-180.
15. Tuckey, R. C., Cheng, C. Y. S. & Slominski, A. T. (2019) The serum vitamin D metabolome: What we know and what is still to discover, J Steroid Biochem Mol Biol. 186, 4-21.
16. Slominski, A. T., Li, W., Kim, T. K., Semak, I., Wang, J., Zjawiony, J. K. & Tuckey, R. C. (2015) Novel activities of CYP11A1 and their potential physiological significance, J Steroid Biochem Mol Biol. 151, 25-37.
17. Slominski, A. T., Kim, T. K., Hobrath, J. V., Janjetovic, Z., Oak, A. S. W., Postlethwaite, A., Lin, Z., Li, W., Takeda, Y., Jetten, A. M. & Tuckey, R. C. (2017) Characterization of a new pathway that activates lumisterol in vivo to biologically active hydroxylumisterols, Sci Rep. 7, 11434.
18. Zhang, L., Lin, D., Sun, X., Curth, U., Drosten, C., Sauerhering, L., Becker, S., Rox, K. & Hilgenfeld, R. (2020) Crystal structure of SARS-CoV-2 main protease provides a basis for design of improved a-ketoamide inhibitors, Science. 368, 409-412.
19. Dai, W., Zhang, B., Jiang, X.-M., Su, H., Li, J., Zhao, Y., Xie, X., Jin, Z., Peng, J. & Liu, F. (2020) Structure-based design of antiviral drug candidates targeting the SARS-CoV-2 main protease, Science. 368, 1331-1335.
20. Naqvi, A. A. T., Fatima, K., Mohammad, T., Fatima, U., Singh, I. K., Singh, A., Atif, S. M., Hariprasad, G., Hasan, G. M. & Hassan, M. I. (2020) Insights into SARS-CoV-2 genome, structure, evolution, pathogenesis and therapies: Structural genomics approach, Biochimica et Biophysica Acta (BBA)-Molecular Basis of Disease, 165878.
21. Yang, H., Yang, M., Ding, Y., Liu, Y., Lou, Z., Zhou, Z., Sun, L., Mo, L., Ye, S. & Pang, H. (2003) The crystal structures of severe acute respiratory syndrome virus main protease and its complex with an inhibitor, Proceedings of the National Academy of Sciences. 100, 13190-13195.
22. Lung, J., Lin, Y. S., Yang, Y. H., Chou, Y. L., Shu, L. H., Cheng, Y. C., Liu, H. T. & Wu, C. Y. (2020) The potential chemical structure of anti-SARS-CoV-2 RNA-dependent RNA polymerase, Journal of medical virology. 92, 693-697.
23. Yin, W., Mao, C., Luan, X., Shen, D.-D., Shen, Q., Su, H., Wang, X., Zhou, F., Zhao, W. & Gao, M. (2020) Structural basis for inhibition of the RNA-dependent RNA polymerase from SARS-CoV-2 by remdesivir, Science. 368, 1499-1504.
24. Mohammad, T., Shamsi, A., Anwar, S., Umair, M., Hussain, A., Rehman, M. T., AlAjmi, M. F., Islam, A. & Hassan, M. I. (2020) Identification of high-affinity inhibitors of SARS-CoV-2 main protease: Towards the development of effective COVID-19 therapy, Virus research. 288, 198102.

Table S1 of Example 1: Structures of sterols and secosteroids tested in this study and the enzymes involved in their production

| | | | Enzyme Involved |
|---|---|---|---|
| No. | Compound Name | Structure | in the synthesis |
| 1 | 24(OH)L3 | 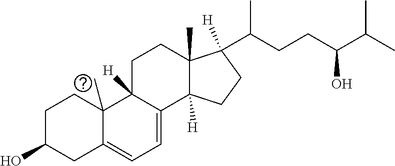 [SMILES: CC(CC[C@H](O)C(C)C)[C@H]1CC[C@H]2C3=CC=C4C[C@@H](O)CCC4(C)[C@@H]3CC[C@]12C] | CYP11A1 |
| 2 | 25(OH)L3 | 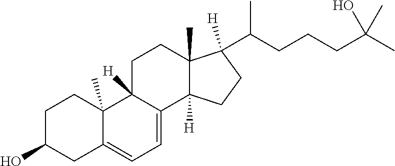 [SMILES: CC(CCCC(C)(C)O)[C@H]1CC[C@H]2C3=CC=C4C[C@@H](O)CC[C@@]4(C)[C@@H]3CC[C@]12C] | CYP27A1 |
| 3 | 22R(OH)7DHC | 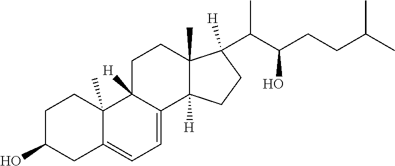 [SMILES: CC(C)CC[C@@H](O)C(C)[C@H]1CC[C@H]2C3=CC=C4C[C@@H](O)CC[C@@]4(C)[C@@H]3CC[C@]12C] | CYP11A1 |
| 4 | 20S(OH)L3 | 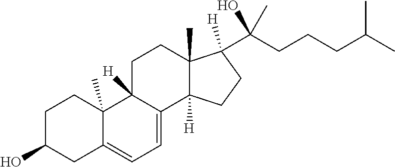 [SMILES: CC(C)CCC[C@](C)(O)[C@H]1CC[C@H]2C3=CC=C4C[C@@H](O)CC[C@@]4(C)[C@@H]3CC[C@@]21C] | CYP11A1 |
| 5 | 25(OH)7DHC | 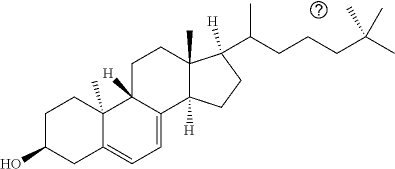 | CYP27A1 |
| 6 | 1,25(OH)₂D3 | 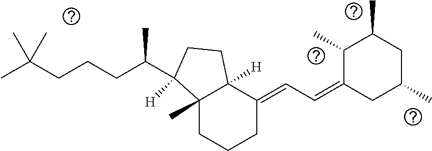 [SMILES: C[C@@H]1C/C(=C/C=C2\CCC[C@]3(C)[C@@H]([C@H](C)CCCC(C)(C)C)CC[C@@H]23)[C@H](C)[C@@H](C)C1] | CYP27B1 CYP2R1 CYP27A1 |
| 7 | 20S(OH)D3 | 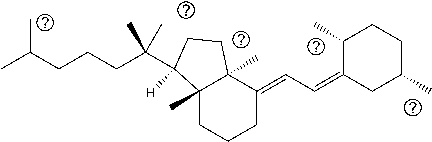 | CYP11A1 |
| 8 | 7DHP | 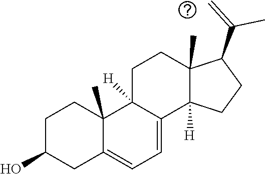 [SMILES: C=C(C)[C@H]1CC[C@H]2C3=CC=C4C[C@@H](O)CC[C@]4(C)[C@H]3CC[C@]12C] | CYP11A1 |
| 9 | 25(OH)D3 | 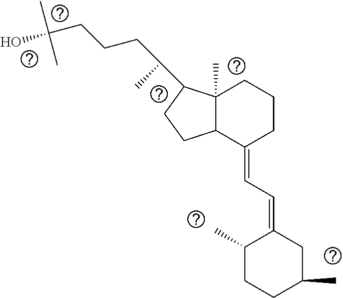 | CYP2R1 CYP27A1 |
| 10 | 20S(OH)7DHC | 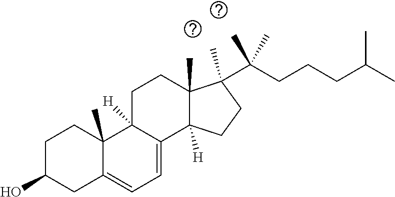 | To be defined |
| 11 | 22R(OH)L3 | 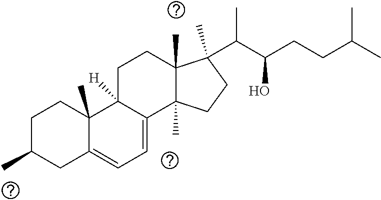 [SMILES: CC(C)CC[C@@H](O)C(C)[C@@]1(C)CC[C@@]2(C)C3=CC=C4C[C@@H](C)CC[C@]4(C)[C@H]3CC[C@@]21C] | CYP11A1 |

25(OH)D3 and 1,25(OH)₂D3 were purchased from Sigma while the other sterols and secosteroids were synthesized enzymatically [1-4] or chemically [5,6].

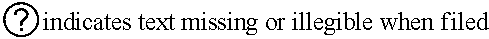

TABLE S2 OF EXAMPLE 1
IC₅₀ values for the inhibition of the enzyme activity of the RdRP by
selected sterols and secosteroids with curcumin used as a standard.

| No. | Compound Name | IC₅₀ (μM) |
|---|---|---|
| 1 | 20(SOH)D3 | 2.6 |
| 2 | 25(OH)D3 | 1.3 |
| 3 | 1,25(OH)2D3 | 1.0 |
| 4 | 20S(OH)L3 | 1.0 |
| 5 | 24(OH)L3 | 2.5 |
| 6 | 25(OH)L3 | 0.5 |
| 7 | 20S(OH)7DHC | 4.7 |
| 8 | Curcumin | 5.1 |

The values for a series of concentrations of each compound were plotted using non-linear regression analysis with a sigmoidal dose-response curve generated as described in the Materials and Methods. The IC₅₀ value was determined as the concentration causing half-maximal activity.

### Example 2

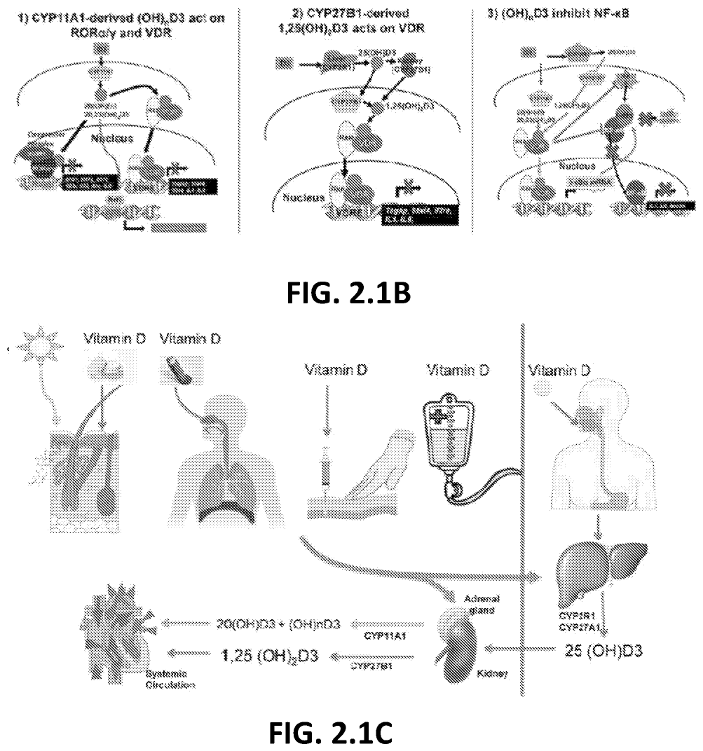

The COVID-19 is currently the foremost health issue in the world. SARS-CoV-2 (severe acute respiratory syndrome coronavirus) is an enveloped positive strain RNA virus in the family Coronaviridae, which also includes the virus SARS-CoV-1 (which was another outbreak in 2002-2003) 1 . COVID-19 has a fatality rate up to —5%, which is several times higher than influenza^{2,3}. The leading cause of death in the patients is due to acute respiratory distress syndrome (ARDS) 2 induced by proinflammatory responses and oxidative stress (FIG. 2.1A).

Vitamin D is a fat-soluble prohormone, which after production in the skin or oral delivery affects important physiological functions in the body including regulation of the innate and adaptive immunity 4-6 . Vitamin D can be activated through canonical and non-canonical pathways (FIG. 2.1A). In the former, it is metabolized to 25-hydroxyvitamin D3 (25(OH)D 3) by CYP2R1 and CYP27A1 in the liver with further metabolism in the kidney to the biologically active 1,25-dihydroxyvitamin D₃ (1,25(OH)₂D 3) by CYP27B1⁷⁻⁹. This metabolism also occurs in a variety of organs, including skin and the immune systemhu7,9.

An alternative pathway of vitamin D activation by CYP11A1 leads to production of more than 10 metabolites some of which are non-calcemic even at high doses^{5,10,11}. These hydroxyderivatives, including 20(OH)D₃ and 20,23(OH)₂D₃,l are produced in humans¹²⁻¹⁵. In addition, 20(OH)D₃ has been detected in the honey, which defines it as a natural product¹⁶. CYP11A1 is expressed not only in adrenals, placenta and gonads but also in immune cells and other peripheral organs¹⁷.

Both 1,25(OH)₂D 3 and non-calcemic CYP11A1 derived metabolites use various, although partially overlapping, mechanisms in enacting their anti-inflammatory and anti-oxidative effects (FIG. 2.1B). 1,25(OH)₂D₃ mediates many of its anti-inflammatory and anti-microbial effects through the vitamin D receptor (VDR) 6,9 . 1,25(OH) 2 D 3 can also inhibit the mitogen-activated protein kinase (MAPK) and NF-kB signaling^{4,9}.

The non-calcemic CYP11A1-derived vitamin D compounds also have their own methods to fight inflammation (FIG. 2.1B). 20(OH)D₃ and their downstream hydroxyderivatives act on VDR as biased agonists^{11,15,19}. They also act as inverse agonists on the retinoic acid-related orphan receptors, RORa and RORy, transcription factors with critical roles in several immune cells and immune responses²⁰⁻²³ (FIG. 2.1B). In addition, CYP11A1-derived derived vitamin D3 derivatives and classical 1,25(OH)₂D 3 can act as agonists on aryl hydrocarbon receptor (AhR)²⁴. Although binding pocket of this receptor can accommodate many different molecules, we believe that secosteroidal signal transduction can be linked to detoxification and anti-oxidative action¹¹ or down-regulation of pro-inflammatory responses²⁵. ARDS and other adverse effects of COVID-19 are induced by cytokine storm

A leading cause of ARDS is “cytokine storm”, a hyperactive immune response triggered by the viral infection (FIG. 2.1A)^{2,26}. It is initiated when the pattern recognition receptor of the innate immune cells recognize the pathogen-associated molecular pattern from a pathogen such as bacteria or virus^{26,27}. The immune cells then release all types of cytokines (interferons, interleukins 1, 6 and 17, chemokines, colony stimulating factors, and tumor necrosis factor (TNF)) leading to hyperinflammation and organ damage²⁷⁻²⁹. In the lungs, alveolar cells are targeted leading to acute lung injury and subsequently ARDS^{27,30}. In severe cases of CoVID-19 other organs and systems are also damaged^{2,3}. Thus, it is crucial to find ways to prevent the “cytokine storm” from going out of control. Although different drugs have been suggested to fight the cytokine storm^{26,27}, they have mixed results and in certain cases can even worsen the disease²⁷. Thus, there is a great need for alternative therapies.

Oxidative stress is also involved in the development of ARDS through action of reactive oxygen species (ROS) and nitrogen species (NRS)³¹⁻³³. The production of ROS and RNS can be triggered by pathogens promoting the secretion of cytokines, which stimulate ROS production thereby producing a positive feedback loop (FIG. 2.1A)^{31,33-35}. Nuclear factor erythroid 2p45-related factor 2 (NRF-2) is a transcription factor that plays a role in the detection of excessive ROS and RNS and induction of mechanisms counteracting the oxidative damage³⁶. NRF-2 loss due to ROS can lead to elevation in proinflammatory cytokine levels and stronger inflammatory responses to stimuli^{31,36}.

### Anti-Inflammatory and Antioxidative Activities of Active Forms of Vitamin D

There is a strong experimental evidence that active forms of vitamin D including the classical 1,25(OH)₂D₃, and novel CYP11A1-derived hydroxyderivatives^{8,11} exert potent anti-inflammatory activities including inhibition of IL-1, IL-6, IL-17, TNFα and INFγ production or other proinflammatory pathways (Supplemental table 1)^{11,18,20,37,38}. The mechanism of action includes downregulation of NF-κB involving action on VDR and inverse agonism on RORγ leading to attenuation of Th17 responses (FIG. 2.1B)^{11,18,20,37-39}. These compounds also induce antioxidative and reparative responses with mechanism of action involving activation of NRF-2 and p53^{11,39-41}.

### Antiviral Effects of Active Forms of Vitamin D

Low vitamin D status in winter permits viral epidemics and vitamin D supplementation could reduce the incidence, severity, and risk of viral diseases⁴²⁻⁴⁶. In addition, several reports have found a strong association between vitamin D deficiency/insufficiency and enhanced COVID-19 severity and mortality⁴⁶⁻⁶³ with the most recent study defining low plasma 25(OH)D₃ as an independent risk factor for COVID-19 infection and hospitalization⁵⁴. Therefore, we retrospectively analyzed microarray data of human epithelial cells treated with 20,23(OH)₂D₃ and 1,25(OH)₂D₃²⁴. We found the downregulation of pathways connected with influenza infection and viral RNA transcription, translation, replication, life cycle and of subject interactions with influenza factors with 20,23(OH)₂D₃ expressing higher anti-viral potency (Table 1).

While 1,25(OH)₂D₃ has the limitation imposed by the toxicity that includes hypercalcemia^{7,9}, CYP11A1-derived 20(OH)D₃, 20(OH)D₂ and 20,23(OH)₂D₃ are not toxic and non-calcemic at very high doses (3-60 μg/kg) at which 1,25(OH)₂D₃ and 25(OH)D₃ are calcemic⁵⁵⁻⁵⁹.

The hyperinduction of proinflammatory cytokines production (cytokine storm), further magnified by oxidative stress induced by the viral infection or cytokines themselves, acting reciprocally in self-amplifying circuitry, gradually damage/destroy the affected organs leading to death in the severe cases of COVID-19 infection (FIG. 2.1A). A solution to the problem fulfilling above premises, are active forms of vitamin D including the classical 1,25(OH)₂D₃ and 25(OH)D₃ (precursors to 1,25(OH)₂D₃)^{5,7,9,45,60} and novel CYP11A1-derived hydroxyderivatives including 20(OH)D₃ and 20,23(OH)₂D₃^{8,11,61}. The former are FDA approved and can immediately be used in the clinic, while the latter are still not approved yet although they fulfill the definition of natural products. They would both terminate “cytokine storm” and oxidative stress with possible anti-viral activity to rescue the patient from the death path (FIG. 2.1). Their preferable routes of delivery are listed in FIG. 2.1C to reach immediately the most affected organs. In this context, active hydroxyforms of vitamin D2 should also be considered^{66,62-64}.

As relates to the vitamin D precursor it is reasonable to propose that patients being admitted with COVID-19 infection to receive as soon as possible 200,000 IU of vitamin D₂ or vitamin D₃ followed by 4,000-10,000 IU/day, if justifiable^{46,66}. Vitamin D3 at 200,000 IU orally has been used to attenuate inflammatory responses induced by the sunburn⁶⁶. It must be noted that application of 250,000-500,000 IU of vitamin D was reported be safe in critically ill patients and was associated with decreased hospital length of stay and improved ability of the blood to carry oxygen (reviewed in^{67,68})

Different routes of delivery of vitamin D precursor can have a profound effect on the final panel of circulating in the body vitamin D derivatives (FIG. 2.1C). Vitamin D delivered orally during the passage through the liver is hydroxylated to 25(OH)D⁷, which is not recognized by CYP11A that only acts on its precursor, vitamin D itself⁶⁹. This likely results in 30 times lower concentration of 20(OH)D₃ in serum in comparison to 25(OH)D₃¹⁴. However, its levels are higher than that of 25(OH)D₃ in the epidermis, a peripheral site of vitamin D3 activation¹⁴. Therefore, adequate systemic (adrenal gland) or local (immune system) production of CYP11A1-derived vitamin D hydroxyderivatives would require parenteral delivery of vitamin D. These routes of vitamin D precursor delivery could include sublingual tablets, intra-muscular, subcutaneous or intravenous injections as well as its aerosolized form of delivery to the lung (FIG. 2.1C). As relates CYP11A1-derived products these would be predominantly generated in the adrenal gland for systemic purposes. However, they can also be generated in peripheral organs expressing CYP11A1 including skin and immune system^{17,70}.

Since vitamin D is readily available, easy to use and relatively nontoxic, it can represent an immediate solution to the problems at relatively high doses, since populations most vulnerable to negative outcome of COVID-19 disease are likely vitamin D deficient and the patients are already under supervision in the hospital environment and are monitored for adverse effects. Vitamin D toxicity is typically not observed until extremely high doses of vitamin D in the range of 50,000-100,000 !Us daily for several months or years⁷¹. Doses up to 500,000 IUs have been routinely given to nursing home patients twice a year in Scandinavian countries to reduce risk for fracture without any evidence of vitamin D intoxication including hypercalcemia, hyperphosphatemia and soft tissue calcification⁷¹.

In addition, we believe that routes of delivery are likely to impact the final outcome, because bypassing liver vitamin D3 will also be accessible to CYP11A1 for metabolism in organs expressing this enzyme.

### References for Example 2

1. Coronaviridae Study Group of the International Committee on Taxonomy of V. The species Severe acute respiratory syndrome-related coronavirus: classifying 2019-nCoV and naming it SARS-CoV-2. Nat Microbiol. 2020;5(4):536-544.
2. Mehta P, McAuley DF, Brown M, et al. COVID-19: consider cytokine storm syndromes and immunosuppression. Lancet. 2020; 395(10229):1033-1034.
3. Zhang C, Shi L, Wang F S. Liver injury in COVID-19: management and challenges. Lancet Gastroenterol Hepatol. 2020.
4. Abhimanyu, Coussens A K. The role of UV radiation and vitamin D in the seasonality and outcomes of infectious disease. Photochem Photobiol Sci. 2017; 16(3):314-338.
5. Wacker M, Holick MF . Sunlight and Vitamin D: A global perspective for health. Dermatoendocrinol. 2013; 5(1):51-108.
6. Dankers W, Colin E M, van Hamburg J P, Lubberts E. Vitamin D in Autoimmunity: Molecular Mechanisms and Therapeutic Potential. Front Immunol 2016; 7:697.
7. Holick M F. Vitamin D deficiency. N Engl J Med. 2007; 357(3):266-281.
8. Tuckey R C, Cheng C Y S, Slominski A T. The serum vitamin D metabolome: What we know and what is still to discover. J Steroid Biochem Mol Biol. 2019; 186:4-21.
9. Bikle D D. Vitamin D: Newer Concepts of Its Metabolism and Function at the Basic and Clinical Level. J Endocr Soc. 2020; 4(2):bvz038.
10. Slominski AT , Kim T K, Li W, Yi A K, Postlethwaite A, Tuckey R C. The role of CYP11A1 in the production of vitamin D metabolites and their role in the regulation of epidermal functions. J Steroid Biochem Mol Biol. 2014; 144 Pt A:28-39.

111. Slominski AT, Chaiprasongsuk A, Janjetovic Z, et al. Photoprotective Properties of Vitamin D and Lumisterol Hydroxyderivatives. Cell Biochem Biophys. 2020; 78(2):165-180.
12. Slominski A T, Kim T K, Shehabi H Z, et al. In vivo evidence for a novel pathway of vitamin D(3) metabolism initiated by P450scc and modified by CYP2761. FASEB J. 2012; 26(9):3901-3915.
13. Slominski A T, Kim T K, Shehabi HZ, et al. In vivo production of novel vitamin D2 hydroxy-derivatives by human placentas, epidermal keratinocytes, Caco-2 colon cells and the adrenal gland. Mol Cell Endocrinol. 2014; 383(1-2):181-192.
14. Slominski AT , Kim T K, Li W, et al. Detection of novel CYP11A1-derived secosteroids in the human epidermis and serum and pig adrenal gland. Sci Rep. 2015; 5:14875.
15. Slominski A T, Kim T K, Li W, Tuckey R C. Classical and non-classical metabolic transformation of vitamin D in dermal fibroblasts. Exp Dermatol. 2016;25(3):231-232.
16. Kim T K, Atigadda V, Brzeminski P, et al. Detection of 7-dehydrocholesterol and vitamin D3 derivatives in honey. Molecules. 2020; Submitted.
17. Slominski R M, Tuckey R C, Manna P R, et al. Extra-adrenal glucocorticoid biosynthesis: implications for autoimmune and inflammatory disorders.Genes lmmun. 2020; 21(3):150-168.
18. Lin Z, Marepally S R, Goh E S Y, et al. Investigation of 20S-hydroxyvitamin D3 analogs and their 1alpha-OH derivatives as potent vitamin D receptor agonists with anti-inflammatory activities. Sci Rep. 2018; 8(1):1478.
19. Kim T K, Wang J, Janjetovic Z, et al. Correlation between secosteroid-induced vitamin D receptor activity in melanoma cells and computer-modeled receptor binding strength. Mol Cell Endocrinol. 2012; 361(1-2):143-152.
20. Slominski A T, Kim T K, Takeda Y, et al. RORalpha and ROR gamma are expressed in human skin and serve as receptors for endogenously produced noncalcemic 20-hydroxy- and 20,23-dihydroxyvitamin D. FASEB J. 2014; 28(7):2775-2789.
21. Jetten A M, Cook D N. (Inverse) Agonists of Retinoic Acid-Related Orphan Receptor gamma: Regulation of Immune Responses, Inflammation, and Autoimmune Disease. Annu Rev Pharmacol Toxicol. 2020; 60:371-390.
22. Slominski A T, Kim T K, Hobrath J V, et al. Endogenously produced nonclassical vitamin D hydroxy-metabolites act as “biased” agonists on VDR and inverse agonists on RORalpha and RORgamma. J Steroid Biochem Mol Biol. 2017; 173:42-56.
23. Jetten A M, Takeda Y, Slominski A, Kang H S. Retinoic acid-related Orphan Receptor gamma (RORgamma): connecting sterol metabolism to regulation of the immune system and autoimmune disease. Curr Opin Toxicol. 2018; 8:66-80.
24. Slominski A T, Kim T K, Janjetovic Z, et al. Differential and Overlapping Effects of 20,23(OH)(2)D3 and 1,25(OH)(2)D3 on Gene Expression in Human Epidermal Keratinocytes: Identification of AhR as an Alternative Receptor for 20,23(OH)(2)D3. Int J Mol Sci. 2018; 19(10).
25. Gutierrez-Vazquez C, Quintana F J. Regulation of the Immune Response by the Aryl Hydrocarbon Receptor. Immunity. 2018; 48(1):19-33.
26. Liu Q, Zhou Y H, Yang Z Q. The cytokine storm of severe influenza and development of immunomodulatory therapy. Cell Mol Immunol. 2016; 13(1):3-10.
27. Tisoncik J R, Korth M J, Simmons C P, Farrar J, Martin T R, Katze M G. Into the eye of the cytokine storm. Microbiol Mol Biol Rev. 2012; 76(1):16-32.
28. Pugin J, Ricou B, Steinberg K P, Suter P M, Martin TR . Proinflammatory activity in bronchoalveolar lavage fluids from patients with ARDS, a prominent role for interleukin-1. Am J Respir Crit Care Med. 1996; 153(6 Pt 1):1850-1856.
29. Chen G, Wu D, Guo W, et al. Clinical and immunologic features in severe and moderate Coronavirus Disease 2019. J Clin Invest. 2020.
30. Kuiken T, Fouchier R A, Schutten M, et al. Newly discovered coronavirus as the primary cause of severe acute respiratory syndrome. Lancet. 2003; 362(9380):263-270.
31. Schieber M, Chandel N S. ROS function in redox signaling and oxidative stress. Curr Biol. 2014; 24(10):R453-462.
32. Ye S, Lowther S, Stambas J. Inhibition of reactive oxygen species production ameliorates inflammation induced by influenza A viruses via upregulation of SOCS1 and SOCS3. J Virol. 2015; 89(5):2672-2683.
33. Vlahos R, Stambas J, Bozinovski S, Broughton B R, Drummond GR, Selemidis S. Inhibition of Nox2 oxidase activity ameliorates influenza A virus-induced lung inflammation. PLoS Pathog. 2011; 7(2):e1001271.
34. To E E, Vlahos R, Luong R, et al. Endosomal NOX2 oxidase exacerbates virus pathogenicity and is a target for antiviral therapy. Nat Commun. 2017; 8(1):69.
35. Imai Y, Kuba K, Neely G G, et al. Identification of oxidative stress and Toll-like receptor 4 signaling as a key pathway of acute lung injury. Cell. 2008; 133(2):235-249.
36. Lee C. Therapeutic Modulation of Virus-Induced Oxidative Stress via the Nrf2-Dependent Antioxidative Pathway. Oxid Med Cell Longev. 2018; 2018:6208067.
37. Janjetovic Z, Tuckey R C, Nguyen M N, Thorpe E M, Jr., Slominski A T. 20,23-dihydroxyvitamin D3, novel P450scc product, stimulates differentiation and inhibits proliferation and NF-kappaB activity in human keratinocytes. J Cell Physiol. 2010; 223(1):36-48.
38. Janjetovic Z, Zmijewski M A, Tuckey R C, et al. 20-Hydroxycholecalciferol, product of vitamin D3 hydroxylation by P450scc, decreases NF-kappaB activity by increasing IkappaB alpha levels in human keratinocytes. PLoS One. 2009; 4(6):e5988.
39. Chaiprasongsuk A, Janjetovic Z, Kim T K, et al. CYP11A1-derived vitamin D3 products protect against UVB-induced inflammation and promote keratinocytes differentiation. Free Radic Biol Med. 2020; 155:87-98.
40. Chaiprasongsuk A, Janjetovic Z, Kim T K, et al. Protective effects of novel derivatives of vitamin D3 and lumisterol against UVB-induced damage in human keratinocytes involve activation of Nrf2 and p53 defense mechanisms. Redox Biol. 2019; 24:101206.
41. Slominski A T, Janjetovic Z, Kim T K, et al. Novel non-calcemic secosteroids that are produced by human epidermal keratinocytes protect against solar radiation. J Steroid Biochem Mol Biol. 2015; 148:52-63.
42. Teymoori-Rad M, Shokri F, Salimi V, Marashi S M. The interplay between vitamin D and viral infections. Reviews in Medical Virology. 2019; 29(2):e2032.
43. Huang F, Zhang C, Liu Q, et al. Identification of amitriptyline HCl, flavin adenine dinucleotide, azacitidine and calcitriol as repurposing drugs for influenza A H5N1 virus-induced lung injury. PLoS Pathog. 2020; 16(3):e1008341.
44. Jakovac H. COVID-19 and vitamin D—Is there a link and an opportunity for intervention? American Journal of Physiology-Endocrinology and Metabolism. 2020; 318(5):E589-E589.
45. Grant W B, Lahore H, McDonnell S L, et al. Evidence that Vitamin D Supplementation Could Reduce Risk of Influenza and COVID-19 Infections and Deaths. Nutrients. 2020; 12(4).
46. Cheng R Z. Can early and high intravenous dose of vitamin C prevent and treat coronavirus disease 2019 (COVID-19)? Med Drug Discov. 2020; 5:100028.
47. D'Avolio A, Avataneo V, Manca A, et al. 25-Hydroxyvitamin D Concentrations Are Lower in Patients with Positive PCR for SARS-CoV-2. Nutrients. 2020; 12(5).
48. Hastie C E, Mackay D F, Ho F, et al. Vitamin D concentrations and COVID-19 infection in UK Biobank. Diabetes Metab Syndr. 2020; 14(4):561-565.
49. Ilie P C, Stefanescu S, Smith L. The role of vitamin D in the prevention of coronavirus disease 2019 infection and mortality. Aging Clin Exp Res. 2020.
50. Jakovac H. COVID-19 and vitamin D-Is there a link and an opportunity for intervention? Am J Physiol Endocrinol Metab. 2020; 318(5):E589.
51. Marik P E, Kory P, Varon J. Does vitamin D status impact mortality from SARS-CoV-2 infection? Med Drug Discov. 2020:100041.
52. McCartney D M, Byrne D G. Optimisation of Vitamin D Status for Enhanced Immuno-protection Against Covid-19. Ir Med J. 2020; 113(4):58.
53. Panarese A, Shahini E. Letter: Covid-19, and vitamin D. Aliment Pharmacol Ther. 2020; 51(10):993-995.
54. Merzon E, Tworowski D, Gorohovski A, et al. Low plasma 25(OH) vitamin D level is associated with increased risk of COVID-19 infection: an Israeli population-based study. The FEBS Joumal.n/a(n/a).
55. Slominski A T, Janjetovic Z, Fuller B E, et al. Products of vitamin D3 or 7-dehydrocholesterol metabolism by cytochrome P450scc show anti-leukemia effects, having low or absent calcemic activity. PLoS One. 2010; 5(3):e9907.
56. Wang J, Slominski A, Tuckey R C, et al. 20-hydroxyvitamin D(3) inhibits proliferation of cancer cells with high efficacy while being non-toxic. Anticancer Res. 2012; 32(3):739-746.
57. Slominski A, Janjetovic Z, Tuckey R C, et al. 20S-hydroxyvitamin D3, noncalcemic product of CYP11A1 action on vitamin D3, exhibits potent antifibrogenic activity in vivo. J Clin Endocrinol Metab. 2013; 98(2):E298-303.
58. Chen J, Wang J, Kim T K, et al. Novel vitamin D analogs as potential therapeutics: metabolism, toxicity profiling, and antiproliferative activity. Anticancer Res. 2014; 34(5):2153-2163.
59. Slominski A T, Kim T K, Janjetovic Z, et al. 20-Hydroxyvitamin D2 is a noncalcemic analog of vitamin D with potent antiproliferative and prodifferentiation activities in normal and malignant cells. Am J Physiol Cell Physiol. 2011; 300(3):C526-541.
60. Shirvani A, Kalajian T A, Song A, Holick M F. Disassociation of Vitamin D′s Calcemic Activity and Non-calcemic Genomic Activity and Individual Responsiveness: A Randomized Controlled Double-Blind Clinical Trial. Sci Rep. 2019; 9(1):17685.
61. Slominski A T, Li W, Kim T K, et al. Novel activities of CYP11A1 and their potential physiological significance. J Steroid Biochem Mol Biol. 2015; 151:25-37.
62. Holick MF. Vitamin D: A millenium perspective. JCell Biochem. 2003; 88(2):296-307.
63. Bikle D D. Vitamin D: an ancient hormone. Exp Dermatol. 2011; 20(1):7-13.
64. Slominski A, Semak I, Wortsman J, et al. An alternative pathway of vitamin D metabolism. Cytochrome P450scc (CYP11A1)-mediated conversion to 20-hydroxyvitamin D2 and 17,20-dihydroxyvitamin D2. FEBS J. 2006; 273(13):2891-2901.
65. Holick M F, Binkley N C, Bischoff-Ferrari H A, et al. Evaluation, treatment, and prevention of vitamin D deficiency: an Endocrine Society clinical practice guideline. J Clin Endocrinol Metab. 2011; 96(7):1911-1930.
66. Scott J F, Das L M, Ahsanuddin S, et al. Oral Vitamin D Rapidly Attenuates Inflammation from Sunburn: An Interventional Study. J Invest Dermatol. 2017; 137(10):2078-2086.
67. Ebadi M, Montano-Loza A J. Perspective: improving vitamin D status in the management of COVID-19. Eur J Clin Nutr. 2020:1-4.

168. Grant W B, Baggerly C A, Lahore H. Reply: “Vitamin D Supplementation in Influenza and COVID-19 Infections. Comment on: Evidence That Vitamin D Supplementation Could Reduce Risk of Influenza and COVID-19 Infections and Deaths Nutrients 2020, 12(4), 988”. Nutrients. 2020; 12(6).
69. Slominski A, Semak I, Zjawiony J, et al. The cytochrome P450scc system opens an alternate pathway of vitamin D3 metabolism. FEBS J. 2005;272(16):4080-4090.
70. Slominski A, Zjawiony J, Wortsman J, et al. A novel pathway for sequential transformation of 7-dehydrocholesterol and expression of the P450scc system in mammalian skin.Eur J Biochem. 2004; 271(21):4178-4188.
71. Holick M F. Vitamin D Is Not as Toxic as Was Once Thought: A Historical and an Up-to-Date Perspective. Mayo Clinic Proceedings. 2015; 90(5):561-564.

Table 1 of Example 2. Gene Set Enrichment Analysis (GSEA) of the microarray
data deposited at NCBI GEO (GSE117351)).
ANTIVIRAL PROPERTIES OF VITAMIN D3-HYDROXYDERIVATIVES

| Reaction | GSEA for 20,23(OH)₂D3 | | | | GSEA for 1,25(OH)₂D3 | | | |
|---|---|---|---|---|---|---|---|---|
| Pathway | NES* | P-value | FDR† | Direction | NES | P-value | FDR | Direction |
| Viral mRNA | −2.818 | 0.00 | 0.012 | Down | −3.601 | 0.00 | 0.00 | Down |
| Translation | | | | | | | | |
| Viral Messenger | −2.513 | 0.00 | 0.013 | Down | −2.405 | 0.00 | 1.860 | Down |
| RNA Synthesis | | | | | | | | |
| Influenza | −3.171 | 0.00 | 3.907 | Down | ^{..} | ^{..} | ^{..} | ^{..} |
| Infection | | | | | | | | |
| Influenza Viral | −3.206 | 0.00 | 3.434 | Down | ^{..} | ^{..} | ^{..} | ^{..} |
| RNA | | | | | | | | |
| Transcription & | | | | | | | | |
| Replication | | | | | | | | |
| Host Interactions | −2.249 | 0.00 | 0.018 | Down | ^{..} | ^{..} | ^{..} | ^{..} |
| with Influenza | | | | | | | | |
| Factors | | | | | | | | |
| HIV Life Cycle | −2.070 | 0.00 | 0.023 | Down | −2.503 | 0.00 | 7.788 | Down |
| Late Phase of | ^{..} | ^{..} | ^{..} | ^{..} | −2.658 | 0.00 | 2.614 | Down |
| HIV Life Cycle | | | | | | | | |
| Host Interactions | −3.340 | 0.00 | 1.354 | Down | ^{..} | ^{..} | ^{..} | ^{..} |
| with HIV factors | | | | | | | | |

*NES—Normalized Enriched Score;
†FDR—False Discovery Rate;
^{..}—the effect is absent

Supplemental Table 1 of Example 2. Comparison of the inhibition of inflammatory gene expression in epidermal keratinocytes by 20,23(OH)₂ D₃ with 1,25(OH)₂ D₃ based on retrospective analysis of microarray data deposited at the NCBI GEO (GSE117351). The values represent relative fold change in the gene expression.

| Gene | 20,23(OH)₂D₃ | 1,25(OH)₂D₃ | Mechanism of action |
|---|---|---|---|
| IL-6 (interleukin 6) | −1.24 | −1.04 | Proinflammatory cytokine |
| AKR1C3 (Aldo-Keto | −12.12 | 1.82 | Downregulation of AKR1C3 is associated with inhibition of |
| Reductase family 1 | | | prostaglandin production (anti-inflammatory, analgesics and |
| member C3; | | | antipyretic action) |
| prostaglandin F | | | |
| synthase) | | | |
| TNF (Tumor necrosis | −1.02 | 1.49 | Pro-inflammatory cytokine |
| factor) | | | |
| TNFRSF14 TNF | −2.95 | 1.01 | Protein involved in inflammatory immune responses |
| Receptor Superfamily | | | |
| Member 14 | | | |
| (Herpesvirus entry | | | |
| mediator) | | | |
| CXCR4 (C-X-C | −6.09 | 1.07 | Receptor for chemotactic activities of lymphocytes |
| chemokine receptor | | | |
| type 4) | | | |
| Il1A (Interleukin 1A) | 3.22 | −1.94 | Cytokine with complex immunoregulatory functions |
| FAT (HLA-F, class I | −1.92 | −1.21 | Protein involved in immune response and development of |
| histocompatibility | | | hepatitis B, type 1 diabetes, systemic lupus erythematosus, |
| antigen, alpha chain Fin | | | hepatocellular carcinoma, involved in MHC class I complex |
| hepatocellular | | | |
| carcinoma) | | | |
| SAA1 (Acute phase | −1.30 | 1.42 | In humans is a plasma biomarker of cardiovascular events |
| isoforms of serum | | | |
| amyloid A) | | | |
| Alox12 (Arachidonate | −1.31 | −1.04 | Alox12 downregulation causes reduction of inflammation and |
| 12-Lipoxygenase) | | | anti-metabolic syndrome, anti-hypertension and anti-obesity |
| | | | properties |
| Alox15B (Arachidonate | −4.42 | −1.52 | Alox15B can regulate cytokines secretion by macrophages; may |
| 15B-Lipoxygenase) | | | also contribute to formation of atherogenic oxLDL and may |
| | | | regulate macrophage differentiation into pro-atherogenic foam |
| | | | cells |
| Alox5 (Arachidonate 5- | −1.01 | 1.03 | Downregulation of Alox5 can cause inhibition of inflammatory |
| Lipoxygenase) | | | response by decrease of leukotriene production, of leucocytes |
| | | | stimulation by chemotactic factors, of allergic reactions, and |
| | | | reduction of asthma and atherosclerosis progression |

### Example 3

Lumisterol and vitamin D derivatives inhibits the hACE2 and SARS-COV2 interactions using commercially available (RBD) kit:

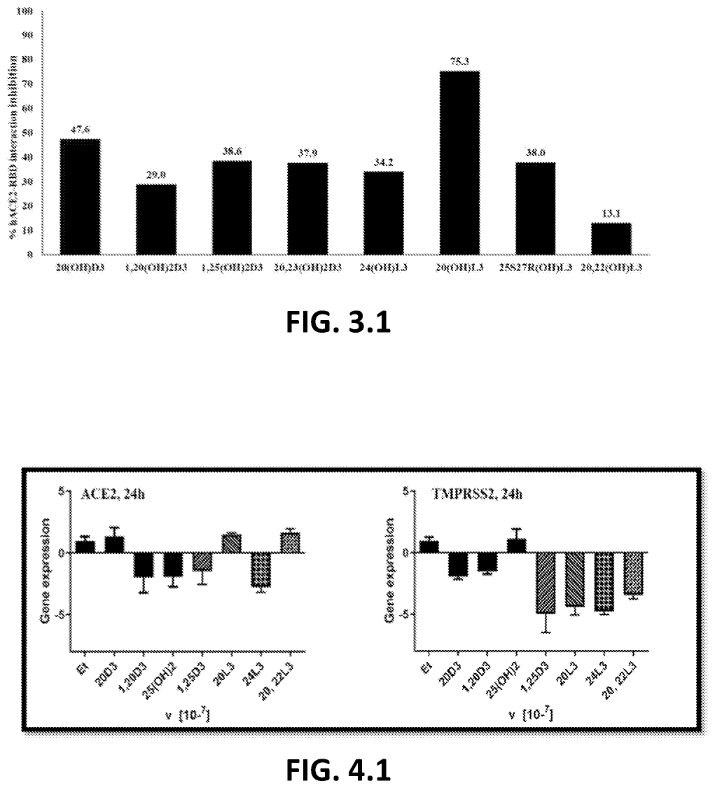

The inventors used the SARS-CoV-2 inhibitor screening kit (EP-105, Acro, Biosystem, USA) to study the inhibition efficiency of selected compounds for hACE2 and SARS-CoV-2 (RBD) interaction. FIG. 3.1 shows inhibition percentage observed in interaction of hACE2 and RBD in presence of the metabolites at concentration of 10⁻⁵ M.

### Example 4

Lumisterol and vitamin D derivatives inhibits the expression of hACE2 in immortalized human HaCaT keratinocytes:

HaCaT keratinocytes were cultured and for RNA isolation the cells were maintained in TPP tissue culture petri dishes (Ø60 mm, 22.1 cm²) in DMEM containing 10% cFBC to reach semiconfluency and then exposed to 10 -7 M compounds or a corresponding concentration of ethanol solvent and were used for RNA isolation (RNAeasy Micro kit, Qiagen) after 24 h of incubation. RNA isolated from HaCaT cells was submitted for cDNA synthesis (High Capacity cDNA Reverse Transcription Kit with RNase Inhibitor, Applied Biosystems) following the manufacturers' protocols. RT-PCR was carried out using Cyber green, in triplicates. CIC-B were used as internal control. Primer sequences are listed as ACE2 (L:TCCAGTACTGTAGATGGTGC (SEQ ID No. 1); R:CTCCTTCTCAGCCTTGTTGC (SEQ ID No. 2)), TMPRSS2 (L;CCTCTTAACAATCCATGGCATTG (SEQ ID No. 3), R;GGGCAGACACACTGGTTTCA SEQ ID No. 4) cyclophilinB (L: TGTGGTGTTTGGCAAAGTTC SEQ ID No. 5; R: GTTTATCCCGGCTGTCTGTC SEQ ID No. 6). Data was analyzed using Graph Pad Prism statistical software one way ANOVA or t-test, where appropriate; *p<0.05; **p<0.01; ***p<0.001. FIG. 4.1 illustrates that lumisterol and vitamin D derivatives inhibit the expression of hACE2 in immortalized human HaCaT keratinocytes.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A pharmaceutical composition, comprising a therapeutically effective amount of an active agent or a pharmaceutically acceptable salt of the active agent, and a pharmaceutically acceptable carrier, to treat a condition, wherein the active agent is selected from the group consisting of: 9β,10α-cholesta-5,7-diene-3β,24-diol, 9β,10α-cholesta-5,7-diene-3β,25-diol, 9β,10α-cholesta-5,7-diene-3β,22-diol, 9β,10αcholesta-5,7-diene-3β,20-diol, cholesta-5,7-dien-3β,20-diol, cholesta-5,7-dien-3β,22-diol, cholesta-5,7-dien-3β,25-diol, 3β-hydroxypregna-5,7-dien-20-one, (3S,5Z,7E)-9,10-secocholesta-5,7,10(19)-trien-3,20-diol, (3S,5Z,7E)-9,10-secocholesta-5,7,10(19)-trien-3,20,23-trio, and optionally(3S,5Z,7E)-9,10-secocholesta-5,7,10(19)-trien-1,3,20-triol, 9β,10α-cholesta-5,7-diene-3β,20,22-triol, and 9β,10α-cholesta-5,7-diene-3β,27-diol, and combinations thereof.

2. The pharmaceutical composition of claim 1, wherein the condition is a COVID-19 condition.

3. The pharmaceutical composition of claim 1, wherein the condition is acute respiratory distress syndrome.

4. The pharmaceutical composition of claim 1, wherein therapeutically effective amount of the COVID-19 agent is a dosage of 2 to 60 microgram/kilogram.

5. The pharmaceutical composition of claim 1, wherein the subject is a human.

6. The pharmaceutical composition of claim 1, wherein the subject is a domesticated animal.

7. The pharmaceutical composition of claim 1, wherein pharmaceutical composition is in the form of an inhalant.

8. The pharmaceutical composition of claim 1, wherein the condition is a viral infection.

9. A method of treating a condition comprising: administering to a subject in need thereof, a pharmaceutical composition, wherein the pharmaceutical composition includes a therapeutically effective amount of an active agent or a pharmaceutically acceptable salt of the active agent, and a pharmaceutically acceptable carrier, wherein the active agent is selected from the group consisting of: 9β,10α-cholesta-5,7-diene-3β,24-diol, 9β,10α-cholesta-5,7-diene-3β,25-diol, 9β,10α-cholesta-5,7-diene-3β,22-diol, 9β,10α-cholesta-5,7-diene-3β,20-diol, cholesta-5,7-dien-3β,20-diol, cholesta-5,7-dien-3β,22-diol, cholesta-5,7-dien-3β,25-diol, 3β-hydroxypregna-5,7-dien-20-one, (3S,5Z,7E)-9,10-secocholesta-5,7,10(19)-trien-3,20-diol, (3S,5Z,7E)-9,10-secocholesta-5,7,10(19)-trien-3,20,23-triol, and optionally(3S,5Z,7E)-9,10-secocholesta-5,7,10(19)-trien-1,3,20-triol, 9β,10α-cholesta-5,7-diene-38,20,22-triol, and 9β,10α-cholesta-5,7-diene-38,27-diol, and combinations thereof.

10. The method of claim 9, wherein the administering is via inhalation.

11. The method of claim 9, wherein the administering is parenteral.

12. The method of claim 1, wherein the condition is a COVID-19 condition.

13. The method of claim 1, wherein the condition is acute respiratory distress syndrome.

14. The method of claim 1, wherein therapeutically effective amount of the active agent is a dosage of 2 to 60 microgram/kilogram.

15. The method of claim 1, wherein the subject is a human.

16. The method of claim 1, wherein the subject is a domesticated animal.

17. The method of claim 1, wherein pharmaceutical composition is in the form of an inhalant.

18. The method of claim 1, wherein the condition is related to HIV, hepatitis, or influenza.

19. The method of claim 1, wherein the condition is a viral infection.

20-36. (canceled)


SEQUENCE LISTING (6 sequences)

SEQ ID NO: 1 - DNA, 20 bp, Artificial Sequence
  Synthesized Forward/left primer for ACE2
tccagtactgtagatggtgc

SEQ ID NO: 2 - DNA, 20 bp, Artificial Sequence
  Synthesized reverse/right primer for ACE2.
ctccttctcagccttgttgc

SEQ ID NO: 3 - DNA, 23 bp, Artificial Sequence
  Synthesized forward/left primer for TMPRSS2
cctcttaacaatccatggcattg

SEQ ID NO: 4 - DNA, 20 bp, Artificial Sequence
  Synthesized reverse/right primer for TMPRSS2
gggcagacacactggtttca

SEQ ID NO: 5 - DNA, 20 bp, Artificial Sequence
  Synthesized forward/left primer sequence for cyclophilinB
tgtggtgtttggcaaagttc

SEQ ID NO: 6 - DNA, 20 bp, Artificial Sequence
  Synthesized reverse/right primer for cyclophilinB
gtttatcccggctgtctgtc